(19) United States
(12) Patent Application Publication
(10) Pub. No.: US 2011/0002885 A1
(43) Pub. Date: Jan. 6, 2011

(54) METHODS OF USING ISOLATED RECOMBINANT POLYPEPTIDE ANTAGONISTS OF SDF-1

(76) Inventors: YONG CHEN, COQUITLAM (CA); GLEB FELDMAN, VANCOUVER (CA); HASSAN SALARI, VANCOUVER (CA)

Correspondence Address: Perkins Coie LLP, P.O. Box 2168, Menlo Park, CA, 94026, US

(21) Appl. No.: 12/883,142

(22) Filed: Sep. 15, 2010

Related U.S. Application Data
(62) Division of application No. 11/725,725, filed on Mar. 20, 2007.
Application No. 11/725,725 is a continuation-in-part of application No. 10/945,674, filed on Sep. 20, 2004, now Pat. No. 7,435,718.
Application No. 10/945,674 is a continuation of application No. 09/852,424, filed on May 9, 2001, now abandoned.
Application No. 09/852,424 is a continuation-in-part of application No. 11/060,031, filed on Feb. 16, 2005, now Pat. No. 7,354,899.
Application No. 11/060,031 is a division of application No. 09/646,193, filed on Mar. 26, 2002, now Pat. No. 6,875,738.
Application No. 09/646,193 is a continuation-in-part of application No. 11/136,097, filed on May 23, 2005, now Pat. No. 7,423,011.
Application No. 11/136,097 is a division of application No. 09/646,192, filed on Mar. 2, 2001, now Pat. No. 6,946,445.
(60) Provisional application No. 60/205,467, filed on May 19, 2000.

(30) Foreign Application Priority Data
Mar. 13, 1998 (CA) 2226391
Aug. 14, 1998 (CA) 2245224
May 9, 2000 (CA) 2305787

(51) Int. Cl. A61K 38/21; A61K 38/16; A61P 35/00; C12N 5/0783

(52) U.S. Cl. 424/85.6; 514/19.2; 435/375

(57) ABSTRACT

This invention is generally directed to a recombinant method of producing SDF-1 receptor antagonists. More particularly, the invention is directed to the isolated and/or recombinant polynucleotide sequences encoding analogs of human SDF-1 alpha or beta and, in particular, SDF-1 analogs having the proline at residue position number 2 replaced with a glycine to provide an SDF-1 receptor antagonist. The recombinant method can be used to produce drugs for a variety of therapeutic uses including, but not limited to, treatment of cancer, inhibiting angiogenesis, and hematopoietic cell proliferation.

### CROSS-REFERENCE TO RELATED APPLICATIONS

This application is a divisional of U.S. patent application Ser. No. 11/725,725, filed May 23, 2007, which is (i) a continuation-in-part of U.S. patent application Ser. No. 10/945,674, filed Sep. 20, 2004, which is a continuation of U.S. patent application Ser. No. 09/852,424, filed May 9, 2001, which claims the benefit of U.S. Provisional Application No. 60/205,467, filed May 19, 2000; wherein, U.S. patent application Ser. No. 10/945,674, filed Sep. 20, 2004, claims the benefit of Canadian Application No. 2305787, filed May 9, 2000;

(ii) a continuation-in-part of U.S. patent application Ser. No. 11/060,031, filed Feb. 16, 2005, which is a divisional of U.S. patent application Ser. No. 09/646,193, filed Mar. 26, 2002, which is a National Stage application of PCT Application No. PCT/CA99/00750, filed Aug. 16, 1999, which claims the benefit of Canadian Application No. 2245224, filed Aug. 14, 1998; and,

(iii) a continuation-in-part of U.S. patent application Ser. No. 11/136,097, filed May 23, 2005, which is a divisional of U.S. patent application Ser. No. 09/646,192, filed Mar. 2, 2001 which is a National Stage application of PCT Application No. PCT/CA99/00221, filed Mar. 12, 1999, which claims the benefit of Canadian Application No. 2226391, filed Mar. 13, 1998, and Canadian Application No. 2245224, filed Aug. 14, 1998;

wherein, each of the applications listed above is hereby incorporated herein by reference in its entirety.

### THE NAMES OF THE PARTIES TO A JOINT RESEARCH AGREEMENT

The claimed invention was made by, on behalf of, and/or in connection with one or more of the following parties to a joint university-corporation research agreement: Chemokine Therapeutics Corp, The University of British Columbia, and British Canadian BioSciences Corp. The agreement was in effect on or before the date the claimed invention was made, and the claimed invention was made as a result of activities undertaken within the scope of the agreement.

### SEQUENCE LISTING

This application contains a paper copy of a sequence listing, the sequence listing in a computer readable format, and a Statement to Support Filing and Submission in accordance with 37 CFR 1.821-1.825.

### BACKGROUND OF THE INVENTION

1. Field of the Invention

This invention is generally directed to a recombinant method of producing SDF-1 receptor antagonists and polynucleotide sequences encoding the antagonists. The method can be used to produce drugs used in a variety of therapeutic applications that include the prevention, treatment, or ameliorization of systems of a variety of diseases.

2. Description of the Related Art

Stromal cell-derived factor-1 (SDF-1) is a member of the chemokine family of structurally related proteins with cell chemoattractant activity. Chemokines (chemoattractant cytokines) are a family of homologous serum proteins of between 7 and 16 kDa, which were originally characterized by their ability to induce migration of leukocytes. Most chemokines have four characteristic cysteines (Cys), and depending on the motif displayed by the first two cysteines, they have been classified into CXC or alpha, CC or beta, C or gamma, and CX3C or delta chemokine classes. Two disulfide bonds are formed between the first and third cysteine and between the second and fourth cysteine. In general, it was thought that the disulfide bridges were required, and Clark-Lewis and co-workers reported that, the disulfide bridges are critical for chemokine activity (Clark-Lewis et al., J. Biol. Chem. 269:16075-16081, 1994). The only exception to having four cysteines is lymphotactin, which has only two cysteine residues. Thus, lymphotactin manages to retain a functional structure with only one disulfide bond. In addition, the CXC, or alpha, subfamily has been divided into two groups depending on the presence of the ELR motif (Glu-Leu-Arg) preceding the first cysteine: the ELR-CXC chemokines and the non-ELR-CXC chemokines (see, e.g., Clark-Lewis, supra, and Belperio et al., “CXC Chemokines in Angiogenesis,” J. Leukoc. Biol. 68:1-8, 2000).

SDF-1 is a 67 amino acid protein in the alpha form and 71 amino acid protein in the beta form and belongs to a group of protein from a super-family of chemo-attractant proteins secreted by a variety of cells including monocytes and lymphocytes as well as other cell types that regulate cell trafficking and immune responses. In humans, the genes of the CXC chemokines are clustered on chromosome 4 (with the exception of SDF-1 gene, which has been localized to chromosome 10) and those of the CC chemokines on chromosome 17. The molecular targets for SDF-1 chemokines are cell surface receptors CXCR4 and CXCR7.

The SDF-1 chemokines are constitutively expressed in lymphoid tissues, indicating that they may have a homeostatic function in regulating lymphocyte trafficking between and within lymphoid organs. However, they are also the main regulators of stem cells growth and differentiation, as well as cancer cells.

The human and mouse SDF-1 predicted protein sequences are approximately 92% identical. Stromal cell derived factor-1α (SDF-1α) and stromal cell derived factor-1β (SDF-1β) are closely related (together referred to herein as SDF-1). The native amino acid sequences of SDF-1α and SDF-1β are known. Identification of genomic clones has shown that the alpha and beta isoforms are a consequence of alternative splicing of a single gene.

Although many chemokines have pro-inflammatory roles, SDF-1 appears to have a fundamental role in the trafficking, export and homing of bone marrow cells. It is produced constitutively, and particularly high levels are found in bone marrow stromal cells. A basic physiological role is implied by the high level of conservation of the SDF-1 sequence between species. In vitro SDF-1 stimulates chemotaxis of a wide range of cells including monocytes and bone marrow-derived progenitor cells. Particularly notable is its ability to stimulate a high percentage of resting and activated T lymphocytes. It is the only known ligand for CXC chemokine receptor 4 (CXCR4), a 7-transmembrane receptor that has been variously described as LESTR, HUMSTR, and fusin. CXCR4 is widely expressed on cells of hemopoietic origin and is a major co-receptor for HIV-1. Consistent with this dual role of CXCR4, SDF-1 blocks HIV-1 entry into CD4+ cells.

The SDF-1 sequence indicates that it belongs to the CXC family of chemokines, but it has only about 22% identity with other chemokines. Despite the divergent primary structure, the recently described three-dimensional structure indicates that it has a similar fold to other chemokines. Furthermore, structure-activity analysis of SDF-1 indicated the importance of N-terminal residues 1-8 for binding and of residues 1 and 2 for receptor activation. Residues 12-17 located in the loop region also contribute to binding. In the SDF-1 structure, the region N-terminal to the CXC motif is highly disordered, but the loop region immediately following the CXC motif is well defined at least in its backbone atoms. These two regions have been identified as being important in other CC and CXC chemokines. As with other chemokines, N-terminal modification of SDF-1 led to dissociation of binding and activity. Thus despite the difference in primary structure, from both a structural and a functional perspective, the general mechanism of receptor binding is similar for SDF-1 and other chemokines.

Peptides corresponding to the N-terminal 1-9 residues of stromal cell-derived factor-1 (SDF-1) have SDF-1 activity. SDF-1 and analogs that consist of residues 1-8, residues 1-9, a dimer of residues 1-9, or residues 1-17 have bound to CXCR4 and induced intracellular calcium and chemotaxis in T lymphocytes and CEM cells. These peptides had similar activities to SDF-1 but were less potent. Whereas native SDF-1 had half-maximal chemoattractant activity at 5 nM, the 1-9 dimer required 500 nM and was therefore 100-fold less potent. The 1-17 and a 1-9 monomer analog were 4- and 36-fold, respectively, less potent than the 1-9 dimer. Both the chemotactic and calcium response of the 1-9 dimer was inhibited by N-terminal modified analog (P2G). The basis for the enhanced activity of the dimer form of SDF-1, 1-9 is uncertain, but it could involve an additional fortuitous binding site on the 1-9 peptide in addition to the normal SDF-1, 1-9 site. A 1-9 analog, 1-9[P2G] dimer, was found to be a CXCR4 antagonist. Further, the 1-67 analog in which the proline the position 2 from the N-terminal side to replaced with glycine (SDF-1 1-67 P2G) was found to be antagonist. Overall this study shows that the N-terminal peptides are CXCR4 agonists or antagonists, and these could be leads for high affinity ligands. Unfortunately, as these analogs were produced using synthetic peptide synthesis, the cost of producing them was high and their activity could be improved, perhaps due to the structure (secondary, tertiary, and quaternary) of the peptide.

Solid tumour growth is generally angiogenesis (neovascularization)-dependent, and angiogenesis inhibitors have therefore been used as agents for the treatment of solid tumours and metastasis. Endothelial cells (EC) in the vasculature play an essential role in angiogenesis, and there is accordingly a need for therapeutic agents that target this activity. The proliferation, migration and differentiation of vascular endothelial cells during angiogenesis are understood to be modulated in both normal and disease states by the complex interactions of a variety of chemokines and chemokine receptors. CXCR4 is expressed on vascular EC, and in such cells is reportedly the most abundant receptor amongst all examined chemokine receptors (Gupta, et al, 1998).

CXCR4 is involved in metastasis. Muller et al. demonstrated a role for CXCR4 in breast cancer metastasis to lymph nodes and lungs. Other results have identified CXCR4 expression in other tumor types such as melanoma; pancreatic, thyroid, renal, and small-cell lung cancers; and squamous cell carcinoma of the tongue. The expression of CXCR4 is associated with decreased survival and increased lymph node metastasis. Blocking the CXCR4 receptor by SDF-1 P2G has been found to prevent metastasis in murine models of breast cancer and malignant melanoma.

Accordingly, one of skill will appreciate novel methods of creating and isolating recombinant SDF-1 antagonists that are useful in a variety of therapeutic applications, particularly recombinant SDF-1 antagonists that can be produced cost-effectively, and have increased activity, perhaps due to the structure of the recombinant polypeptide.

### SUMMARY OF THE INVENTION

The invention is generally directed to a recombinant SDF-1 receptor antagonist and the polynucleotide encoding the antagonist. In many embodiments, the invention is directed to an isolated and/or recombinant polypeptide comprising SEQ ID NO:1; or an amino acid sequence that is at least 95% homologous to SEQ ID NO:1, conserves the Gly at residue position number 2, and binds to an SDF-1 receptor. The invention can also include an isolated and/or recombinant polynucleotide comprising a nucleotide sequence that encodes for this polypeptide, such as SEQ ID NO:2. In some embodiments, the invention can include a vector comprising such a polynucleotide or a host cell transformed by such a vector.

In some embodiments, the isolated and/or recombinant polypeptide comprising SEQ ID NO:1; or an amino acid sequence that is at least 95% homologous to SEQ ID NO:1, conserves the Gly at residue position number 2, and binds to an SDF-1 receptor, can further comprise additional amino acid residues. In some embodiments, the peptide can further comprise the sequence Lys-Arg-Phe-Lys (SEQ ID NO:33) at the C-terminus to provide a polypeptide comprising SEQ ID NO:3 or an amino acid sequence that is at least 95% homologous to SEQ ID NO:3, conserves the Gly at residue position number 2, and binds to an SDF-1 receptor. The invention can also include an isolated and/or recombinant polynucleotide comprising a nucleotide sequence that encodes for this polypeptide, such as SEQ ID NO:4. The invention can also include an isolated and/or recombinant polynucleotide comprising a nucleotide sequence that encodes for this polypeptide, such as SEQ ID NO:2. In some embodiments, the invention can include a vector comprising such a polynucleotide or a host cell transformed by such a vector.

The invention can be directed to a method of preparing the polypeptides described above, comprising culturing a host cell under conditions suitable to produce the desired polypeptide; and recovering the polypeptide from the host cell culture; wherein, the host cell comprises an exogenously-derived polynucleotide encoding the desired polypeptide. In some embodiments, the host cell is E. Coli. In some embodiments, the polypeptide is a fusion polypeptide having an affinity tag, and the recovering includes (1) capturing and purifying the fusion polypeptide, and (2) removing the affinity tag for high yield production of the desired polypeptide or an amino acid sequence that is at least 95% homologous to desired polypeptide, conserves the Gly at residue position number 2, and binds to a CXCR7 receptor.

The invention can be directed to a method of decreasing the activity of an SDF-1 receptor comprising contacting the receptor with the desired polypeptide and, in some embodiments, the receptor is a CXCR7 receptor. The invention can be directed to a method of inhibiting interferon gamma production by an activated T-cell comprising contacting the activated T-cell with the desired polypeptide and, in some embodiments, the activated T-cell is a human T-lymphoma cell. In some embodiments, the method further comprises contacting the activated T-cell with interferon beta to provide a synergistic down-regulation of interferon gamma production.

The invention can be directed to a method of increasing hematopoietic cell proliferation comprising contacting a hematopoietic cell with the desired polypeptide and, in some embodiments, the hematopoietic cell is a bone marrow progenitor cell. In some embodiments, the invention can be directed to a method of increasing hematopoietic cell proliferation in a subject by administering to the subject a therapeutically effective amount of the desired polypeptide in a pharmaceutically acceptable carrier and, in some embodiments, the hematopoietic cell is a bone marrow progenitor cell.

The invention can be directed to a method of inhibiting the growth of a solid tumor in a subject by administering to the subject a therapeutically effective amount of the desired polypeptide in a pharmaceutically acceptable carrier and, in some embodiments, the solid tumor is lung carcinoma.

As such, the invention can also be directed to a method of inhibiting angiogenesis in a subject by administering to the subject a therapeutically effective amount of the desired polypeptide in a pharmaceutically acceptable carrier and, in some embodiments, the inhibiting includes reducing neovascularization of a solid tumor.

### BRIEF DESCRIPTION OF THE DRAWINGS

FIG. 1 illustrates the restriction enzyme map of the synthetic DNA for rhSDF-1 alpha P2G according to some embodiments of the present invention.

FIG. 2 illustrates the plasmid map of the expression vector for rhSDF-1 alpha P2G (6×His tag disclosed as SEQ ID NO:34) according to some embodiments of the present invention.

FIG. 3 illustrates an amino acid alignment of rhSDF-1alpha P2G (residues 1-68 of SEQ ID NO:3) with human native mature SDF-1alpha (SEQ ID NO:31) according to some embodiments of the present invention.

FIG. 4 illustrates the restriction enzyme map of the synthetic DNA for rhSDF-1 beta P2G according to some embodiments of the present invention.

FIG. 5 illustrates the plasmid map of the expression vector for rhSDF-1 beta P2G (6×His tag disclosed as SEQ ID NO:34) according to some embodiments of the present invention.

FIG. 6 illustrates an amino acid alignment of rhSDF-1 beta P2G (SEQ ID NO:36) with human native mature SDF-1 beta (SEQ ID NO:35) according to some embodiments of the present invention.

FIGS. 7A and 7B show purification of the rhSDF-1alpha P2G and rhSDF-1beta P2G, respectively, using Coomassie Blue staining and 15% SDS-PAGE analyses according to some embodiments of the present invention.

FIG. 8 shows competitive CXCR4 receptor binding between SDF-1 and rhSDF-1alpha P2G on a CEM cell line according to some embodiments of the present invention.

FIG. 9 shows competitive CXCR4 receptor binding between SDF-1 and rhSDF-1alpha P2G on an RDF-1 cell line according to some embodiments of the present invention.

FIG. 10 shows the effect of induction of calcium mobilization by rhSDF-1alpha P2G analogs at a concentration of 1 μm according to some embodiments of the present invention.

### DETAILED DESCRIPTION OF THE INVENTION

The embodiments of the present invention are generally directed to a recombinant SDF-1 receptor antagonist and the polynucleotide encoding the antagonist, and these antagonists can be used, for example, in a variety of therapeutic applications including, but not limited to, prevention, treatment, and/or ameliorization of symptoms of disease.

Many of the terms taught herein can be construed according to what is known to one of skill in the art. The terms “treat” and “treatment,” for example, can be used interchangeably with the terms “prevention” and “ameliorization of symptoms” in some embodiments. As such, “treatment” can include, but is not limited to, obtaining beneficial or desired clinical results, such as alleviation of symptoms; diminishment of the extent of a disease; stabilizing a disease condition; delaying or slowing the progression of a disease; ameliorating or palliating symptoms of a disease; and partial or total remission, regardless of whether the remission is detectable or undetectable. “Treatment” may also refer to therapeutic and prophylactic measures; as well as to prolonging the survival of a patient. A subject that is in “need” of a method taught herein includes subjects that already have a disease as well as those in which the onset of a disease may be prevented. The term “subject” and “patient” can be used interchangeably and refer to an animal such as a mammal including, but not limited to, non-primates such as, for example, a cow, pig, horse, cat, dog, rat and mouse; and primates such as, for example, a monkey or a human.

SDF-1 includes two isoforms: stromal cell-derived factor-1α (SDF-1α) (SEQ ID NO:31) and stromal cell-derived factor-1β (SDF-1β) (SEQ ID NO:32). The human CXC chemokine SDF-1, for example, can be defined as having a total of 67 amino acid residues as shown below:

| (residues 1-67 of SEQ ID NO: 31) | |
|---|---|
| Lys¹-Pro-Val-Ser-Leu-Ser-Tyr-Arg-Cys-Pro-Cys-Arg-Phe-Phe¹⁴-Glu- | 15 |
| Ser-His-Val-Ala-Arg-Ala-Asn-Val-Lys-His-Leu-Lys-Ile-Leu-Asn- | 30 |
| Thr-Pro-Asn-Cys-Ala-Leu-Gln-Ile-Val-Ala-Arg-Leu-Lys-Asn-Asn- | 45 |
| Asn-Arg-Gln-Val-Cys-Ile-Asp-Pro-Lys-Leu⁵⁵-Lys-Trp-Ile-Gln-Glu- | 60 |
| Tyr-Leu-Glu-Lys-Ala-Leu-Asn⁶⁷ | 67 |

The amino acids are identified in the present application by the following conventional three-letter abbreviations:

| | | |
|---|---|---|
| Alanine | A | Ala |
| Arginine | R | Arg |
| Asparagine | N | Asn |
| Aspartic acid | D | Asp |
| Cysteine | C | Cys |
| Glutamic acid | E | Glu |
| Glutamine | Q | Gln |
| Glycine | G | Gly |
| Histidine | H | His |
| Isoleucine | I | Ile |
| Ornithine | O | Orn |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| Other | | Xaa |

The single letter identifier is provided for ease of reference. The three-letter abbreviations are generally accepted in the peptide art, recommended by the IUPAC-IUB commission in biochemical nomenclature, and are required by WIPO Standard ST.25. Furthermore, the peptide sequences are taught according to the generally accepted convention of placing the N-terminus on the left and the C-terminus on the right of the sequence listing as required by WIPO Standard ST.25.

SDF-1 is functionally distinct from other chemokines in that it plays a fundamental role in the trafficking, export and homing of bone marrow progenitor cells, as well as in the regulation of stem cell and angioblast activity. These activities of SDF-1, such as the regulation of hematopoietic stem cells, can be exploited to produce agents that are highly useful in a variety of therapeutic applications such as the treatment of cancer, inhibition of angiogenesis, and hematopoietic cell proliferation.

The SDF-1 Analogs

The teachings herein are based on the discovery that mutated forms of SDF-1 alpha and beta can be recombinantly produced to provide useful antagonists of SDF-1 for a variety of therapeutic applications. These antagonists can not only serve as antagonists to their respective form of SDF-1, but also as antagonists to other forms of SDF-1. For example, in some embodiments, the alpha form can act as an antagonist to the beta form, the beta form can act as an antagonist to the alpha form, and interspecies antagonist behavior is also contemplated. In some embodiments, the antagonists can be referred to interchangeably as analogs, mimetics, mutants, recombinants, polypeptides of the present invention. Most any form of these terms, modified or unmodified, can also be used interchangeably including, but not limited to, “recombinant polypeptide mutant,” “recombinant analog,” “recombinant SDF-1 chemokine analog,” and the like. The biological activity of the analogs can be referenced using any measures known and accepted to one of skill including, but not limited to, receptor binding, chemotaxis, calcium mobilization, and the like.

The SDF-1 analogs taught herein are useful for treating or preventing inflammatory conditions, autoimmune disorders, cancer, graft rejection, bacterial infection, viral infection, vascular conditions (for example, atherosclerosis, restenosis, systemic lupus erythematosis, and ischemia-reperfusion), sepsis, tumorigenesis, and angiogenesis; stem cell mobilization as well as vaccine production and blood cell recovery following chemotherapy. Inflammatory conditions contemplated by the present invention include both acute and chronic inflammatory diseases. The mutants may also prove useful in conducting gene therapy; one manner they may assist in the methods of gene therapy is through an arrest of the cell cycle.

In some embodiments, the uses of the mutants include, but are not limited to, treatment or management of arthritis, asthma, colitis/illeitis, psoriasis, atherosclerosis and the like. In some embodiments, the mutants can be used to treat or manage autoimmune conditions include, but are not limited to, rheumatoid arthritis, multiple sclerosis and other autoimmunological diseases. In some embodiments, the mutants can be used to treat or manage cancer include, but are not limited to, treatment or management of human malignancy/cancer cell metastasis and relapses. In some embodiments, the mutants can be used to modulate in blood cell recovery include, but are not limited to, blood cell elevation after chemotherapy/radiotherapy and stem cell mobilization for transplantations. In some embodiments, the mutants can be used for vaccine production include, but are not limited to, enhancement in humoral antibody production, increases in antigen presenting T-cells, increases in dendritic cells and immunological features known as vaccine induction. In some embodiments, the mutants can be used to treat osteoporosis and, in other embodiments to treat genetic diseases through gene therapy.

In many embodiments, the invention is directed to an isolated and/or recombinant polypeptide that is a mutant of SDF-1 alpha or beta. The polypeptide can comprise SEQ ID NO:1; or an amino acid sequence that is at least 95% homologous to SEQ ID NO:1, conserves the Gly at residue position number 2, and binds to an SDF-1 receptor. The invention can also include an isolated and/or recombinant polynucleotide comprising a nucleotide sequence that encodes for this polypeptide, such as SEQ ID NO:2. In some embodiments, the invention can include a vector comprising such a polynucleotide or a host cell transformed by such a vector.

The term “isolated” means altered “by the hand of man” from its natural state; i.e., if it occurs in nature, it has been changed or removed from its original environment, or both. For example, a naturally occurring polynucleotide or a polypeptide naturally present in a living animal in its natural state is not “isolated,” but the same polynucleotide or polypeptide separated from the coexisting materials of its natural state is “isolated”, as the term is employed herein. For example, with respect to polynucleotides, the term isolated means that it is separated from the chromosome and cell in which it naturally occurs. However, a nucleic acid molecule contained in a clone that is a member of a mixed clone library (e.g., a genomic or cDNA library) and that has not been isolated from other clones of the library (e.g., in the form of a homogeneous solution containing the clone without other members of the library) or a chromosome isolated or removed from a cell or a cell lysate (e.g., a “chromosome spread”, as in a karyotype), is not “isolated” for the purposes of this invention. Moreover, a nucleic acid molecule contained in a preparation of mechanically or enzymatically cleaved genomic DNA is also not “isolated” for the purposes of this invention. As part of or following isolation, such polynucleotides can be joined to other polynucleotides, for mutagenesis, to form fusion proteins, and for propagation or expression in a host, for instance. The isolated polynucleotides, alone or joined to other polynucleotides such as vectors, can be introduced into host cells, in culture or in whole organisms, after which such DNAs still would be isolated, as the term is used herein, because they would not be in their naturally occurring form or environment. Similarly, the polynucleotides and polypeptides may occur in a composition, such as a media formulations, solutions for introduction of polynucleotides or polypeptides, for example, into cells, compositions or solutions for chemical or enzymatic reactions, for instance, which are not naturally occurring compositions, and, therein remain isolated polynucleotides or polypeptides within the meaning of that term as it is employed herein

A “vector,” such as an expression vector, is used to transfer or transmit the DNA of interest into a prokaryotic or eukaryotic host cell, such as a bacteria, yeast, or a higher eukaryotic cell. Vectors can be recombinantly designed to contain a polynucleotide encoding a desired polypeptide. These vectors can include a tag, a cleavage site, or a combination of these elements to facilitate, for example, the process of producing, isolating, and purifying the polypeptide. The DNA of interest can be inserted as the expression component of a vector. Examples of vectors include plasmids, cosmids, viruses, and bacteriophages. If the vector is a virus or bacteriophage, the term vector can include the viral/bacteriophage coat. The term “expression vector” is usually used to describe a DNA construct containing gene encoding an expression product of interest, usually a protein, that is expressed by the machinery of the host cell. This type of vector is frequently a plasmid, but the other forms of expression vectors, such as bacteriophage vectors and viral vectors (e.g., adenoviruses, replication defective retroviruses, and adeno-associated viruses), can be used.

In some embodiments, the isolated and/or recombinant polypeptide comprising SEQ ID NO:1; or an amino acid sequence that is at least 95% homologous to SEQ ID NO:1, conserves the Gly at residue position number 2, and binds to an SDF-1 receptor, can further comprise additional amino acid residues. In some embodiments, the peptide can further comprise the sequence Lys-Arg-Phe-Lys (SEQ ID NO:33) at the C-terminus to provide a polypeptide comprising SEQ ID NO:3 or an amino acid sequence that is at least 95% homologous to SEQ ID NO:3, conserves the Gly at residue position number 2, and binds to an SDF-1 receptor. The invention can also include an isolated and/or recombinant polynucleotide comprising a nucleotide sequence that encodes for this polypeptide, such as SEQ ID NO:4. The invention can also include an isolated and/or recombinant polynucleotide comprising a nucleotide sequence that encodes for this polypeptide, such as SEQ ID NO:2. In some embodiments, the invention can include a vector comprising such a polynucleotide or a host cell transformed by such a vector.

Methods of Preparing the Recombinant SDF-1 Polynucleotide and Polypeptide Mutants

The invention can be directed to a method of preparing the polypeptides described above, comprising culturing a host cell under conditions suitable to produce the desired polypeptide; and recovering the polypeptide from the host cell culture; wherein, the host cell comprises an exogenously-derived polynucleotide encoding the desired polypeptide. In some embodiments, the host cell is E. Coli.

Initially, a double-stranded DNA fragment encoding the primary amino acid sequence of recombinant polypeptide mutant of human stromal cell-derived factor 1 α/β (rhSDF-1α/β) is designed. This DNA fragment is manipulated to facilitate synthesis, cloning, expression or biochemical manipulation of the expression products. The synthetic gene is ligated to a suitable cloning vector and then the nucleotide sequence of the cloned gene is determined and confirmed. The gene is then amplified using designed primers having specific restrict enzyme sequences introduced at both sides of insert gene, and the gene is subcloned into a suitable subclone/expression vector. The expression vector bearing the synthetic gene for the mutant is inserted into a suitable expression host. Thereafter the expression host is maintained under conditions suitable for production of the gene product, and the protein is isolated and purified from the cells expressing the gene.

The nucleic acid (e.g., cDNA or genomic DNA) mutants may be inserted into a replicable vector for cloning (amplification of the DNA) for expression. Various vectors are publicly available. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence.

The signal sequence may be a prokaryotic signal sequence selected, for example, from the group of the alkaline phosphatase, penicillinase, Ipp, or heat-stable enterotoxin II leaders. For yeast secretion the signal sequence may be, e.g., the yeast invertase leader, alpha factor leader (including Saccharomyces and Kluyveromyces alpha-factor leaders, the latter described in U.S. Pat. No. 5,010,182), or acid phosphatase leader, the C. albicans glucoamylase leader (EP 362,179), or the signal described in WO 90/13646. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most. Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take the encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 (Stinchcomb et al., Nature, 282:39 (1979); Kingsman et al., Gene, 7:141 (1979); Tschemper et al., Gene, 10:157 (1980)). The trpl gene provides a selection marker for a mutant strain of yeast lacking the ability to grow in tryptophan, for example, ATCC No. 44076 or PEP4-1 (Jones, Genetics, 85:12 (1977)).

Expression and cloning vectors usually contain a promoter operably linked to the encoding nucleic acid sequence to direct mRNA synthesis. Promoters recognized by a variety of potential host cells are well known. Promoters suitable for use with prokaryotic hosts include the .beta.-lactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980); EP 36,776), and hybrid promoters such as the tac promoter (deBoer et al., Proc. Natl. Acad. Sci. USA, 80:21 25 (1983)). Promoters for use in bacterial systems also will contain a. Shine-Dalgarno sequence operably linked to the encoding DNA.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

PRO87299 transcription from vectors in mammalian host cells is controlled, for example, by promoters obtained from the genomes of viruses such as polyoma virus, fowlpox virus (UK 2,211,504), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of the encoding DNA by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, a-fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the vector at a position 5′ or 3′ to the coding sequence but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the mutants.

In some embodiments, the expression control sequence is selected from a group consisting of a lac system, T7 expression system, major operator and promoter regions of pBR322 origin, and other prokaryotic control regions. Still other methods, vectors, and host cells suitable for adaptation to the synthesis of the mutants in recombinant vertebrate cell culture are described in Gething et al., Nature, 293:620 625 (1981); Mantei et al., Nature, 281:40 46 (1979); EP 117,060; and EP 117,058.

The mutants can be expressed as a fusion protein, adding a number of amino acids to the protein, and in some embodiments to the amino terminus of the protein. These extra amino acids can serve as affinity tags or cleavage sites, for example. Fusion proteins are usually designed to: (1) assist in purification by acting as a temporary ligand for affinity purification, (2) produce a precise recombinant by removing extra amino acids using a cleavage site between the target gene and affinity tag, (3) increase the solubility of the product, and (4) increase expression of the product. A proteolytic cleavage site is often included at the junction of the fusion region and the protein of interest to enable further purification of the product-separation of the recombinant protein from the fusion protein following affinity purification of the fusion protein. Such enzymes, and their cognate recognition sequences, can include Factor. Xa, thrombin and enterokinase, cyanogen bromide, trypsin, or chymotrypsin, for example. Typical fusion expression vectors include pGEX (Pharmacia Biotech Inc; Smith, D. B. and Johnson, K. S. Gene 67:31-40 (1988)), pMAL (New England. Biolabs, Beverly, Mass.), pRIT5 (Pharmacia, Piscataway, N.J.), and pET (Strategen), which fuse glutathione S-transferase (GST), maltose E binding protein, protein A, or six histidine (His) (SEQ ID NO:34), respectively, to the target recombinant protein.

Synthetic DNAs containing the sequences of nucleotides, tags and cleavage sites can be designed and provided as a modified coding for recombinant polypeptide mutants of human stromal cell-derived factor 1 α/β (rhSDF-1α/β). For example, six histidine sequences (SEQ ID NO:34) comprising his tag for affinity purification can be preferred in some embodiments.

In some embodiments, the polypeptide is a fusion polypeptide having an affinity tag, and the recovering step includes (1) capturing and purifying the fusion polypeptide, and (2) removing the affinity tag for high yield production of the desired polypeptide or an amino acid sequence that is at least 95% homologous to desired polypeptide, conserves the Gly at residue position number 2, and binds to a CXCR7 receptor. In some embodiments, the fusion polypeptide is selected from a group consisting of SEQ ID NOs. 5, 7, 9, 11, 14, 16, 18, and 20. In some embodiments, the encoding polynucleotide for the fusion polypeptide is selected from a group consisting of SEQ ID NOs. 6, 8, 10, 12, 15, 17, 19, and 21.

In some embodiments, the expression vectors are the plasmids of SEQ ID NOs:13 or 22. SEQ ID NO:22, for example, encodes for the fusion polypeptide of SEQ ID NO:14 having an affinity tag with six histidines (SEQ ID NO:34) and a. Met site for cyanogen bromide-mediated cleavage. Likewise, SEQ ID NO: 13, for example, encodes for the fusion polypeptide SEQ ID NO:5 having an affinity tag with six histidines (SEQ ID NO:34) and a Met site for cyanogen bromide-mediated cleavage. SEQ ID NO:8, for example, encodes for SEQ ID NO:7 having an affinity tag with six histidines (SEQ ID NO:34) and a. Factor Xa cleavage site. SEQ ID NO:10, for example, encodes for SEQ ID NO:9 having an affinity tag with six histidines (SEQ ID NO:34) and an enterokinase cleavage site. SEQ ID NO:12, for example, encodes for SEQ ID NO:11 having an extra Met residue at the N-terminus. SEQ ID NO 17, for example, encodes SEQ ID NO:16 having an affinity tag with six histidines (SEQ ID NO:34) and a Factor Xa cleavage site. SEQ ID NO:19, for example, encodes for SEQ ID NO:18 having an affinity tag with six histidines (SEQ ID NO:34) and an enterokinas cleavage site. SEQ ID NO: 21, for example, encodes for SEQ ID NO:20 having an extra Met residue at the N-terminus.

DNA encoding the mutants may be obtained from a cDNA library prepared from tissue possessing the mRNA for the mutants. As such, the DNA can be conveniently obtained from a cDNA library prepared from human tissue. The encoding gene for the mutants may also be obtained from a genomic library or by known synthetic procedures (e.g., automated nucleic acid synthesis).

Libraries can be screened with probes (such as antibodies to the mutant P2G of human stromal cell derived factor 1 alpha or oligonucleotides of at least about 20-80 bases) designed to identify the gene of interest or the protein encoded by it. Screening the cDNA or genomic library with the selected probe may be conducted using standard hybridization procedures, such as described in Sambrook et al., Molecular Cloning: A Laboratory Manual (New York: Cold Spring Harbor Laboratory Press, 1989), which is herein incorporated by reference. An alternative means to isolate the gene encoding recombinant polypeptide mutants of human stromal cell-derived factor 1 α/β (rhSDF-1α/β) is to use PCR methodology [Sambrook et al., supra; Dieffenbach et al., PCR Primer: A Laboratory Manual (Cold Spring Harbor Laboratory Press, 1995)].

Nucleic acids having a desired protein coding sequence may be obtained by screening selected cDNA or genomic libraries using a deduced amino acid sequence and, if necessary, a conventional primer extension procedure as described in Sambrook et al., supra, to detect precursors and processing intermediates of mRNA that may not have been reverse-transcribed into cDNA.

In some embodiments, an isolated nucleotide sequence will hybridizable, under moderately stringent conditions, to a nucleic acid having a nucleotide sequence comprising or complementary to the desirednucleotide sequences. In some embodiments, an isolated nucleotide sequence will hybridizable, under stringent conditions, to a nucleic acid having a nucleotide sequence comprising or complementary to the desirednucleotide sequences. A nucleic acid molecule is “hybridizable” to another nucleic acid molecule when a single-stranded form of the nucleic acid molecule can anneal to the other nucleic acid molecule under the appropriate conditions of temperature and ionic strength (see Sambrook et al., supra,). The conditions of temperature and ionic strength determine the “stringency” of the hybridization. “Hybridization” requires that two nucleic acids contain complementary sequences. However, depending on the stringency of the hybridization, mismatches between bases may occur. The appropriate stringency for hybridizing nucleic acids depends on the length of the nucleic acids and the degree of complementation. Such variables are well known in the art. More specifically, the greater the degree of similarity or homology between two nucleotide sequences, the greater the value of Tm for hybrids of nucleic acids having those sequences. For hybrids of greater than 100 nucleotides in length, equations for calculating Tm have been derived (see Sambrook et al., supra). For hybridization with shorter nucleic acids, the position of mismatches becomes more important, and the length of the oligonucleotide determines its specificity (see Sambrook et al., supra).

The terms “homology” and “homologous” can be used interchangeably in some embodiments. The terms can refer to nucleic acid sequence matching and the degree to which changes in the nucleotide bases between polynucleotide sequences affects the gene expression. These terms also refer to modifications, such as deletion or insertion of one or more nucleotides, and the effects of those modifications on the functional properties of the resulting polynucleotide relative to the unmodified polynucleotide. Likewise the terms refer to polypeptide sequence matching and the degree to which changes in the polypeptide sequences, such as those seen when comparing the modified polypeptides to the unmodified polypeptide, affect the function of the polypeptide. It should appreciated to one of skill that the polypeptides, such as the mutants taught herein, can be produced from two non-homologous polynucleotide sequences within the limits of degeneracy.

In some embodiments, the polynucleotides of the present invention are at least 80, 85, 90, or 95 percent homologous to the desired polynucleotide or any degenerate form of the desired polynucleotide. In some embodiments, the polypeptides of the present invention are at least 80, 85, 90, or 95 percent homologous to the desired polypeptide. In some embodiments, the polypeptide is at least 85, 90, or 95 percent homologous to the desired polypeptide and binds to an SDF-1 receptor. In some embodiments the polypeptide is 95 percent homologous to rhSDF-1 alpha or beta P2G.

The selection of expression vectors, control sequences, transformation methods, and the like, are dependent on the type of host cell used to express the gene. Following entry into a cell, all or part of the vector DNA, including the insert DNA, may be incorporated into the host cell chromosome, or the vector may be maintained extrachromosomally. Those vectors that are maintained extrachromosomally are frequently capable of autonomous replication in the host cell. Other vectors are integrated into the genome of a host cell upon and are replicated along with the host genome.

Host cells are transfected or transformed with the expression or cloning vectors described herein to produce the mutants. The cells are cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra, each of which are incorporated by reference.

The host cells can be prokaryotic or eukaryotic and, suitable host cells for cloning or expressing the DNA in the vectors herein can include prokaryote, yeast, or higher eukaryote cells. Methods of eukaryotic cell transfection and prokaryotic cell transformation are known to the ordinarily skilled artisan, for example, CaCl2, CaPO4, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456 457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527 537 (1990) and Mansour et al., Nature, 336:348 352 (1988).

Suitable host cells for cloning or expressing the DNA in the vectors herein include prokaryote, yeast, or higher eukaryote cells. Suitable prokaryotes include, but are not limited to, eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772(ATCC 53,635). Other suitable prokaryotic host cells include Enterobacteriaceae such as Escherichia, e.g., E. coli, Enterobacter, Erwinia, Klebsiella, Proteus, Salinonella,e.g., Salmonella typhimunrium, Serratia, e.g., Serratia marcescans, and Shigella, as well as Bacilli such as B. subtilis and B. lichenifonnis (e.g., B. lichenifonnis 41 P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1 A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac) 169 degP ompT kanr; E. coli W3110 strain 37D6, which has the complete genotype tonA ptr3 phoA E15 (argF-lac) 169 degP ompT rbs7 ilvC kanr; E. coli W3110 strain 40B4, which is 37D6 with a non-kanamycin resistant degP deletion mutation; and an E. coli strain having mutant periplasmic protease as disclosed in U.S. Pat. No. 4,946,783. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for the mutants. Saccharomyces cerevisiae is a commonly used lower eukaryotic host microorganism. Others include Schizosaccharomyces pombe (Beach and Nurse, Nature, 290: 140 (1981); EP 139,383 published 2 May 1985); Kluyveromyces hosts (U.S. Pat. No. 4,943,529; Fleer et al., Bio/Technology, 9:968 975 (1991)) such as, e.g., K. lactis (MW98-8C, CBS683, CBS4574; Louvencourt et al., J. Bacteriol., 154(2):737 742 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265 278 [1988]); Candida; Trichoderma reesia (EP 244,234); Neurospora crassa (Case et al., Proc. Natl. Acad. Sci. USA, 76:5259 5263 (1979)); Schwanniomyces such as Schwanniomyces occidentalis (EP 394,538); and filamentous fungi such as, e.g., Neurospora, Penicillium, Tolypocladium (WO 91/00357), and Aspergillus hosts such as A. nidulans (Ballance et al., Biochem. Biophys. Res. Commun., 112:284 289 (1983); Tilburn et al., Gene, 26:205 221 (1983); Yelton et al., Proc. Natl. Acad. Sci. USA, 81: 1470 1474 (1984)) and A. niger (Kelly and Hynes, EMBO J., 4:475 479 (1985)) Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated mutants are derived from multicellular organisms. Invertebrate cells include insect cells such as Drosophila S2 and Spodoptera Sf9, as well as plant cells. Useful mammalian host cell lines include Chinese hamster ovary (CHO) and COS cells. More specific examples include monkey kidney CVI line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/−DHFR (CHO, Urlaub and Chasin, Proc. Natl. Acad. Sci. USA, 77:4216 (1980)); mouse sertoli cells (TM4, Mather, Biol. Reprod., 23:243 251 (1980)); human lung cells (W138, ATCC CCL 75); human liver cells (Hep G2, HB 8065); and mouse mammary tumor (MMT 060562, ATCC CCL5 1). One of skill can readily choose the appropriate host cell without undue experimentation.

The mutants can be variants of the rhSDF-1 alpha or beta P2G polypeptides. The term “variant” refers to modifications to a peptide that allows the peptide to retain its binding properties, and such modifications include, but are not limited to, conservative substitutions in which one or more amino acids are substituted for other amino acids; deletion or addition of amino acids that have minimal influence on the binding properties or secondary structure; conjugation of a linker; post-translational modifications such as, for example, the addition of functional groups. Examples of such post-translational modifications can include, but are not limited to, the addition of modifying groups described below through processes such as, for example, glycosylation, acetylation, phosphorylation, modifications with fatty acids, formation of disulfide bonds between peptides, biotinylation, PEGylation, and combinations thereof. The mimetics may be created by either directly or indirectly connecting at least one modifying group to a reactive group on the mimetic. In fact, in most embodiments, the mutants can be modified with any of the various modifying groups known to one of skill.

The term “modifying group” refers to any chemical moiety that can be attached to a reactive site on a polypeptide taught herein, such as a radioactive label, diagnostic label, biolabel, poly(ethylene glycol) (PEG), etc., composing a portion of a mimetic that was either absent in the native chemokine or that comprises an isolated sequence of less than four amino acids. Examples of reactive sites that can serve as sites for modification include, but are not limited to, an amino group such as the alpha-amino group at the amino-terminus of a peptide; a carboxyl group at the carboxy-terminus of a peptide; a hydroxyl group such as those present on a tyrosine, serine or threonine residue; or, any other suitable reactive group on an amino acid side chain.

In some embodiments, the modifying group can include a codrug. In some embodiments, the modifying group can include a glycosaminoglycan such as heparin or hirudin, a biochemical label, an N-terminal modifier capable of reducing the ability of the SDF-1 mimetic to act as a substrate for aminopeptidases, and a C-terminal modifier capable of reducing the ability of the SDF-1 mimetic to act as a substrate for carboxypeptidases.

In some embodiments, the modifying group may also be a “biotinyl structure”, which includes biotinyl groups and analogues and derivatives thereof. Examples of biotinyl structures include, but are not limited to, iminiobiotinyl structures such as, for example, a 2-iminobiotinyl group. The modifications, for example, may control the pharmacokinetic or pharmacodynamic properties of a mimetic without substantially reducing its bioactive function, or alter in vivo stability, bioavailability, or half-life of the mimetic. In some embodiments, the modifications can provide a diagnostic capability such as, for example, by creating a means of detecting the presence or location of a mimetic in vivo or in vitro

In some embodiments, an amine group of an SDF-1 mimetic can be combined with a carboxyl-terminated PEG (Nektar Corp.) in the presence of, for example, EDC or DCC to form a pegylated structure through formation of an amide bond between the SDF-1 mimetic and the PEG. In some embodiments, either a succinimidyl derivative of mPEG (Nektar Corp.) or an isocyanate-terminated mPEG (Nektar Corp.) can be combined with an SDF-1 mimetic under conditions known to those of skill in the art. In another example, the carboxyl group of an SDF-1 mimetic can be activated with, for example, EDC or DCC and combined with an amino-terminated mPEG (Nektar Corp.) In some embodiments, an amine group of an SDF-1 mimetic can be combined with a methacrylate-terminated mPEG (Nektar Corp.) in the presence of an initiator capable of undergoing thermal or photolytic free radical decomposition. Examples of suitable initiators include benzyl-N,N-diethyldithiocarbamate or p-xylene-N,N-diethyldithiocarbamate. The molecular weights of the PEG moieties can range from about 500 Daltons to about 40,000 Daltons, from about 500 Daltons to about 20,000 Daltons, or any range therein.

In some embodiments, the modifying groups can include, but are not limited to, O-modified derivatives including, but not limited to, C-terminal hydroxymethyl benzyl ether, and other C-terminal hydroxymethyl derivatives; N-modified derivatives including, but not limited to, substituted amides such as alkylamides; hydrazides and compounds in which a C-terminal phenylalanine residue is replaced with a phenethylamide analogue such as, for example, by replacing Ser-lle-Phe with Ser-lle-phenethylamide.

In some embodiments, the modifying groups may include a fluorescein-containing group. Examples of fluorescein-containing groups include, but are not limited to, 5-(and 6-)-carboxyfluorescein succinimidyl ester and fluorescein isothiocyanate. In some embodiments, the modifying group may include a cholyl structure. An example of a cholyl derivative is 3-(O-aminoethyl-iso)-cholyl (Aic).

In some embodiments, the modifying group may include N-acetylneuraminyl, trans-4-cotininecarboxyl, 2-imino-1-imidazolidineacetyl, (S)-(−)-indoline-2-carboxyl, 2-norbornaneacetyl, γ-oxo-5-acenaphthenebutyryl, (−)-2-oxo-4-thiazolidinecarboxyl group, tetrahydro-3-furoyl group, 4-morpholinecarbonyl group, 2-thiopheneacetyl group, 2-thiophenesulfonyl group, diethylene-triaminepentaacetyl group, (O)-methoxyacetyl group, N-acetylneuraminyl group, and combinations thereof. In some embodiments, the modifying groups may include light scattering groups, magnetic groups, nanogold, other proteins, a solid matrix, radiolabels, carbohydrates, and combinations thereof.

In some embodiments, the modifying groups can include diagnostic agents such as, for example, materials that are radiopaque, radioactive, paramagnetic, fluorescent, lumiscent, and detectable by ultrasound. In some embodiments, the radiopaque agents are materials comprising iodine or iodine-derivatives such as, for example, iohexyl and iopamidol. In some embodiments, the radioactive materials are radioisotopes, which can be detected by tracing radioactive emissions. Examples of radioactive materials include, but are not limited to, ¹⁴C, ¹²³I, ¹²⁴I, ¹²⁵I, ^{99m}Tc, ³⁵S or ³H.

In many embodiments, the molecular weight of an agent connected to a mimetic should be at or below about 40,000 Daltons, or any range therein, to ensure elimination of the agent from a subject. In some embodiments, the molecular weight of the agent ranges from about 300 Daltons to about 40,000 Daltons, from about 8,000 Daltons to about 30,000 Daltons, from about 10,000 Daltons to about 20,000 Daltons, or any range therein. It is to be appreciated that one skilled in the art should recognize that some of the groups, subgroups, and individual modifying groups taught herein may not be used in some embodiments of the present invention.

The variants can be merely conservatively modified variants of the rhSDF-1 alpha or beta P2G polypeptides containing only conservative substitutions. The term “conservatively modified variant” refers to a conservative amino acid substitution, which is an amino acid substituted by an amino acid of similar charge density, hydrophilicity/hydrophobicity, size, and/or configuration such as, for example, substituting valine for isoleucine. In comparison, a “non-conservatively modified variant” refers to a non-conservative amino acid substitution, which is an amino acid substituted by an amino acid of differing charge density, hydrophilicity/hydrophobicity, size, and/or configuration such as, for example, substituting valine for phenyalanine.

As described above, one of skill will understand that the mutants have a variety of therapeutic uses. For example, the invention can be directed to a method of decreasing the activity of an SDF-1 receptor comprising contacting the receptor with the desired polypeptide and, in some embodiments, the receptor is a CXCR4 receptor or a CXCR7 receptor.

The term “contacting” refers to placing an agent, such as a compound of the present invention in contact with a cellular receptor, and this placing can occur in vivo, ex vivo, in situ, or in vitro. In some embodiments, the contacting can include adding an SDF-1 mimetic to a liquid medium containing a cell, and the liquid medium may also contain a solvent, such as dimethyl sulfoxide (DMSO), to facilitate the uptake of the mimetic into the cell. In some embodiments, the contacting can include administering an SDF-1 mimetic to a subject in need, wherein the administering can be performed using any method taught herein, such as, for example, direct injection to a target tissue. Without intending to be bound by any theory or mechanism of action, the cellular receptors that can be activated by the mimetic of the present invention include CXCR4 and CXCR7, or a combination thereof.

The invention can be directed to a method of inhibiting interferon gamma production by an activated T-cell comprising contacting the activated T-cell with the desired polypeptide and, in some embodiments, the activated T-cell is a human T-lymphoma cell. In some embodiments, the method further comprises contacting the activated T-cell with interferon beta to provide a synergistic down-regulation of interferon gamma production. See U.S. Pat. Nos. 6,875,738 and 6,946,445, each of which is hereby incorporated by reference herein in its entirety.

Hematopoietic cells include, for example, primitive bone marrow progenitor cells and stem cells. Hematopoietic cells that are uncommitted to a final differentiated cell type are identified herein as “progenitor” cells. Hematopoietic progenitor cells possess the ability to differentiate into a final cell type directly or indirectly through a particular developmental lineage. Undifferentiated, pluripotent progenitor cells that are not committed to any lineage are referred to herein as “stem cells.” All hematopoietic cells can in theory be derived from a single stem cell, which is also able to perpetuate the stem cell lineage, as daughter cells become differentiated. The isolation of populations of mammalian bone marrow cell populations which are enriched to a greater or lesser extent in pluripotent stem cells has been reported (see for example, C. Verfullie et al., J. Exp. Med., 172, 509 (1990), which is incorporated herein by reference. Bone marrow transplantation has been used in the treatment of a variety of hematological, autoimmune and malignant diseases. In conjunction with bone marrow transplantation, ex vivo hematopoietic (bone marrow) cell culture may be used to expand the population of hematopoietic cells, particularly progenitor or stem cells, prior to reintroduction of such cells into a patient. In ex vivo gene therapy, hematopoietic cells may be transformed in vitro prior to reintroduction of the transformed cells into the patient. In gene therapy, using conventional recombinant DNA techniques, a selected nucliec acid, such as a gene, may be isolated, placed into a vector, such as a viral vector, and the vector transfected into a hematopoietic cell, to transform the cell, and the cell may in turn express the product coded for by the gene. The cell then may then be introduced into a patient. Hematopoietic stem cells were initially identified as a prospective target for gene therapy (see e.g., Wilson, J. M., et al., Proc. Natl. Acad. Sci. 85: 3014-3018 (1988)). However, problems have been encountered in efficient hematopoietic stem cell transfection (see Miller, A. D., Blood 76: 271-278 (1990)). There is accordingly a need for agents and methods that facilitate the proliferation of hematopoietic cells in ex vivo cell culture. There is also a need for agents that may be used to facilitate the establishment and proliferation of engrafted hematopoietic cells that have been transplanted into a patient. See U.S. application Ser. No. 10/945,674, which is hereby incorporated by reference herein in its entirety.

A further application of CXCR4 antagonists is cancer therapy. For example, the growth of solid tumors is angiogenesis-dependent, and the endothelial cells (essential for the blood vessels formation) carry the SDF-1 receptor, so SDF-1 antagonists will inhibit tumor growth through anti-angiogenesis effect. Accordingly, the invention can be directed to a method of inhibiting the growth of a solid tumor in a subject by administering to the subject a therapeutically effective amount of the desired polypeptide in a pharmaceutically acceptable carrier and, in some embodiments, the solid tumor is lung carcinoma. See U.S. Pat. Nos. 6,875,738 and 6,946,445, each of which is hereby incorporated by reference herein in its entirety.

As such, the invention can be directed to a method of inhibiting angiogenesis in a subject by administering to the subject a therapeutically effective amount of the desired polypeptide in a pharmaceutically acceptable carrier and, in some embodiments, the inhibiting includes reducing neovascularization of a solid tumor. See U.S. Pat. Nos. 6,875,738 and 6,946,445, each of which is hereby incorporated by reference herein in its entirety.

Pharmaceutical Compositions

The invention further provides pharmaceutical compositions containing the mimetics. The pharmaceutical compositions include a mimetic in an amount that is diagnostic, therapeutic and/or prophylactic in the diagnosis, prevention, treatment and amelioration of symptoms of disease.

The amount of a mimetic used in the compositions can vary according to factors such as type of disease, age, sex, and weight of the subject. Dosage regimens may be adjusted to optimize a therapeutic response. In some embodiments, a single bolus may be administered; several divided doses may be administered over time; the dose may be proportionally reduced or increased; or any combination thereof, as indicated by the exigencies of the therapeutic situation and factors known one of skill in the art. It is to be noted that dosage values may vary with the severity of the condition to be alleviated. Dosage regimens may be adjusted over time according to the individual need and the professional judgment of the person administering or supervising the administration of the compositions, and the dosage ranges set forth herein are exemplary only and do not limit the dosage ranges that may be selected by medical practitioners.

The terms “administration” or “administering” refer to a method of incorporating a compound into the cells or tissues of a subject, either in vivo or ex vivo to diagnose, prevent, treat, or ameliorate a symptom of a disease. In one example, a compound can be administered to a subject in vivo parenterally. In another example, a compound can be administered to a subject by combining the compound with cell tissue from the subject ex vivo for purposes that include, but are not limited to, cell expansion and mobilization assays. When the compound is incorporated in the subject in combination with one or active agents, the terms “administration” or “administering” can include sequential or concurrent incorporation of the compound with the other agents such as, for example, any agent described above. A pharmaceutical composition of the invention is formulated to be compatible with its intended route of administration. Examples of routes of administration include, but are not limited to, parenteral such as, for example, intravenous, intradermal, intramuscular, and subcutaneous injection; oral; inhalation; intranasal; transdermal; transmucosal; and rectal administration.

An “effective amount” of a compound of the invention can be used to describe a therapeutically effective amount or a prophylactically effective amount. A “therapeutically effective amount” refers to an amount that is effective at the dosages and periods of time necessary to achieve a desired therapeutic result and may also refer to an amount of active compound, prodrug or pharmaceutical agent that elicits any biological or medicinal response in a tissue, system, or subject that is sought by a researcher, veterinarian, medical doctor or other clinician that may be part of a treatment plan leading to a desired effect.

The therapeutically effective amount may need to be administered in an amount sufficient to result in amelioration of one or more symptoms of a disorder, prevention of the advancement of a disorder, or regression of a disorder. In some embodiments, a therapeutically effective amount can refer to the amount of a therapeutic agent that improves a subject's condition by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

A “prophylactically effective amount” refers to an amount that is effective at the dosages and periods of time necessary to achieve a desired prophylactic result. Typically, a prophylactic dose is used in a subject prior to the onset of a disease, or at an early stage of the onset of a disease, to prevent or inhibit onset of the disease or symptoms of the disease. A prophylactically effective amount may be less than, greater than, or equal to a therapeutically effective amount.

In some embodiments, the administration can be oral. In some embodiments, the administration can be subcutaneous injection. In some embodiments, the administration can be intravenous injection using a sterile isotonic aqueous buffer. In some embodiments, the administration can include a solubilizing agent and a local anesthetic such as lignocaine to ease discomfort at the site of injection. In some embodiments, the administrations may be parenteral to obtain, for example, ease and uniformity of administration.

The compounds can be administered in dosage units. The term “dosage unit” refers to discrete, predetermined quantities of a compound that can be administered as unitary dosages to a subject. A predetermined quantity of active compound can be selected to produce a desired therapeutic effect and can be administered with a pharmaceutically acceptable carrier. The predetermined quantity in each unit dosage can depend on factors that include, but are not limited to, (a) the unique characteristics of the active compound and the particular therapeutic effect to be achieved, and (b) the limitations inherent in the art of creating and administering such dosage units.

A “pharmaceutically acceptable carrier” is a diluent, adjuvant, excipient, or vehicle with which the mimetic is administered. A carrier is pharmaceutically acceptable after approval by a state or federal regulatory agency or listing in the U.S. Pharmacopeial Convention or other generally recognized sources for use in subjects. The pharmaceutical carriers include any and all physiologically compatible solvents, dispersion media, coatings, antibacterial and antifungal agents, isotonic and absorption delaying agents, and the like. Examples of pharmaceutical carriers include, but are not limited to, sterile liquids, such as water, oils and lipids such as, for example, phospholipids and glycolipids. These sterile liquids include, but are not limited to, those derived from petroleum, animal, vegetable or synthetic origin such as, for example, peanut oil, soybean oil, mineral oil, sesame oil, and the like. Water can be a preferred carrier for intravenous administration. Saline solutions, aqueous dextrose and glycerol solutions can also be liquid carriers, particularly for injectable solutions.

Suitable pharmaceutical excipients include, but are not limited to, starch, sugars, inert polymers, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain minor amounts of wetting agents, emulsifying agents, pH buffering agents, or a combination thereof. The compositions can take the form of solutions, suspensions, emulsion, tablets, pills, capsules, powders, sustained-release formulations and the like. Oral formulations can include standard carriers such as, for example, pharmaceutical grades mannitol, lactose, starch, magnesium stearate, sodium saccharine, cellulose, magnesium carbonate, and the like. See Martin, E. W. Remington's Pharmaceutical Sciences. Supplementary active compounds can also be incorporated into the compositions.

In some embodiments, the carrier is suitable for parenteral administration. In some embodiments, the carrier can be suitable for intravenous, intraperitoneal, intramuscular, sublingual or oral administration. In some embodiments, the pharmaceutically acceptable carrier may comprise pharmaceutically acceptable salts, such as acid addition salts. For purposes of the present invention, the term “salt” and “pharmaceutically acceptable salt” can be used interchangeably in most embodiments. Pharmaceutically acceptable salts are non-toxic at the concentration in which they are administered and include those salts containing sulfate, hydrochloride, phosphate, sulfonate, sulfamate, sulfate, acetate, citrate, lactate, tartrate, methanesulfonate, ethanesulfonate, benzenesulfonate, p-toluenesulfonate, cyclohexylsulfonate, cyclohexylsulfamate, and quinate. Pharmaceutically acceptable salts can be obtained from acids, such as hydrochloric acid, sulfuric acid, phosphoric acid, sulfonic acid, sulfonic acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfonic acid, cyclohexylsulfamic acid, and quinic acid. Such salts can be prepared, for example, by reacting the free acid or base form of the product with one or more equivalents of the desired base or acid in a solvent in which the salt is insoluble, or in water that is later removed using a vacuum. Ion exchange can also be used to prepare desired salts.

Pharmaceutical formulations for parenteral administration may include liposomes. Liposomes and emulsions are delivery vehicles or carriers that are especially useful for hydrophobic drugs. Depending on biological stability of the therapeutic reagent, additional strategies for protein stabilization may be employed. Furthermore, one may administer the drug in a targeted drug delivery system such as, for example, in a liposome coated with target-specific antibody. The liposomes will bind to the target protein and be taken up selectively by the cell expressing the target protein.

Therapeutic compositions typically must be sterile and stable under the conditions of manufacture and storage. The composition can be formulated as a solution, microemulsion, liposome, or other ordered structure suitable for a high drug concentration. In some embodiments, the carrier can be a solvent or dispersion medium including, but not limited to, water; ethanol; a polyol such as for example, glycerol, propylene glycol, liquid polyethylene glycol, and the like; and, combinations thereof. The proper fluidity can be maintained in a variety of ways such as, for example, using a coating such as lecithin, maintaining a required particle size in dispersions, and using surfactants.

In some embodiments, isotonic agents can be used such as, for example, sugars; polyalcohols that include, but are not limited to, mannitol, sorbitol, glycerol, and combinations thereof; and sodium chloride. Sustained absorption characteristics can be introduced into the compositions by including agents that delay absorption such as, for example, monostearate salts, gelatin, and slow release polymers. Carriers can be used to protect active compounds against rapid release, and such carriers include, but are not limited to, controlled release formulations in implants and microencapsulated delivery systems. Biodegradable and biocompatible polymers can be used such as, for example, ethylene vinyl acetate, polyanhydrides, polyglycolic acid, collagen, polyorthoesters, polylactic acid, polycaprolactone, polyglycolic copolymer (PLG), and the like. Such formulations can generally be prepared using methods known to one of skill in the art.

Local administration of the mimetics to a target tissue, particular in diseases that include ischemic tissue, can be used in the methods taught herein. In some embodiments, the mimetics are administered by injections that can include intramuscular, intravenous, intra-arterial, intracoronary, intramyocardial, intrapericardial, intraperitoneal, subcutaneous, intrathecal, or intracerebrovascular injections.

The compounds may be administered as suspensions such as, for example, oily suspensions for injection. Lipophilic solvents or vehicles include, but are not limited to, fatty oils such as, for example, sesame oil; synthetic fatty acid esters, such as ethyl oleate or triglycerides; and liposomes. Suspensions that can be used for injection may also contain substances that increase the viscosity of the suspension such as, for example, sodium carboxymethyl cellulose, sorbitol, or dextran. Optionally, a suspension may contain stabilizers or agents that increase the solubility of the compounds and allow for preparation of highly concentrated solutions.

In one embodiment, a sterile and injectable solution can be prepared by incorporating an effective amount of an active compound in a solvent with any one or any combination of desired additional ingredients described above, filtering, and then sterilizing the solution. In another embodiment, dispersions can be prepared by incorporating an active compound into a sterile vehicle containing a dispersion medium and any one or any combination of desired additional ingredients described above. Sterile powders can be prepared for use in sterile and injectable solutions by vacuum drying, freeze-drying, or a combination thereof, to yield a powder that can be comprised of the active ingredient and any desired additional ingredients. Moreover, the additional ingredients can be from a separately prepared sterile and filtered solution. In another embodiment, a mimetic may be prepared in combination with one or more additional compounds that enhance the solubility of the mimetic.

In some embodiments, the compounds can be administered by inhalation through an aerosol spray or a nebulizer that may include a suitable propellant such as, for example, dichlorodifluoromethane, trichlorofluoromethane, dichlorotetrafluoroethane, carbon dioxide, or a combination thereof. In one example, a dosage unit for a pressurized aerosol may be delivered through a metering valve. In another embodiment, capsules and cartridges of gelatin, for example, may be used in an inhaler and can be formulated to contain a powderized mix of the compound with a suitable powder base such as, for example, starch or lactose.

In some embodiments, a therapeutically or prophylactically effective amount of a mimetic may range in concentration from about 0.001 nM to about 0.1 M; from about 0.001 nM to about 0.05 M; from about 0.01 nM to about 15 μM; from about 0.01 nM to about 10 μM, or any range therein. In some embodiments, the mimetics may be administered in an amount ranging from about 0.001 mg/kg to about 50 mg/kg; from about 0.005 mg/kg to about 40 mg/kg; from about 0.01 mg/kg to about 30 mg/kg; from about 0.01 mg/kg to about 25 mg/kg; from about 0.1 mg/kg to about 20 mg/kg; from about 0.2 mg/kg to about 15 mg/kg; from about 0.4 mg/kg to about 12 mg/kg; from about 0.15 mg/kg to about 10 mg/kg, or any range therein, wherein a human subject is assumed to average about 70 kg.

The mimetics of the present invention can be administered as a diagnostic, therapeutic or prophylactic agent in a combination therapy with the administering of one or more other agents. The agents of the present invention can be administered concomitantly, sequentially, or cyclically to a subject. Cycling therapy involves the administering a first agent for a predetermined period of time, administering a second agent for a second predetermined period of time, and repeating this cycling for any desired purpose such as, for example, to enhance the efficacy of the treatment. The agents of the present invention can also be administered concurrently. The term “concurrently” is not limited to the administration of agents at exactly the same time, but rather means that the agents can be administered in a sequence and time interval such that the agents can work together to provide additional benefit. Each agent can be administered separately or together in any appropriate form using any appropriate means of administering the agent or agents.

Each of the agents described herein can be administered to a subject in combination therapy. In some embodiments, the agents can be administered at points in time that vary by about 15 minutes, 30 minutes, 1 hour, 2 hours, 4 hours, 8 hours, 12 hours, 18 hours, 24 hours, 48 hours or 1 week in time. In some embodiments, at least one of the agents is an immunomodulatory agent. In some embodiments, the agents can include antiproliferatives, antineoplastics, antimitotics, anti-inflammatories, antiplatelets, anticoagulants, antifibrins, antithrombins, antibiotics, antiallergics, antioxidants, and any prodrugs, codrugs, metabolites, analogs, homologues, congeners, derivatives, salts and combinations thereof.

The present invention encompasses sustained release formulations for the administration of one or more agents. In some embodiments, the sustained release formulations can reduce the dosage and/or frequency of the administrations of such agents to a subject.

### EXAMPLES

The following examples illustrate, but do not limit, the present invention.

### Example 1

The expression vectors described herein can have a fusion tag (CBD-tag, Intein-tag, or GST-tag) at the N-terminal for affinity purification to produce a high purity of recombinant. Target recombinants can be released from fusion tags by a simple cleavage reaction mediated by factor Xa, enterokonese or cyanogen bromide, or a leading peptide self-release. A final recombinant can be produced without extra amino acids. In addition, a construct can be engineered for expression of SDF-1 mutants without any tagging, resulting in a final recombinant having, for example, one extra amino acid of Methionine at the N-terminal. The expression vectors developed using the methods taught herein were sequenced for final confirmation.

Prior to protein production, each recombinant is verified using tests that include Western-blotting, mass spectrometry, sequence identification, and assays of biological activity in the processing steps. The processing can include gene cloning, protein induction, expression, isolation, cleavage from an affinity tag, and final purification.

Cloning optimization strategies included altering expression vector to have a suitable cleavage site and fusion tag to improve yield and purity of the product. For example, an optimal expression system may produce recombinants in an inclusion body for preventing protease digestion within cells, having a leader peptide for self cleavage, and a 6-his tag (SEQ ID NO:34) for high yield protein induction and easy affinity-purification in both natural and denatured conditions without any tag for direct expression.

### Example 2

Cloning of rhSDF-1-P2G cDNA into a subclone vector: rhSDF-1alpha P2G DNA sequence (SEQ ID NO: 1) was designed, synthesized, and cloned into plasmid pUC57 with restriction enzyme EcoR V at two sites. The DNA sequence was confirmed by sequence analysis. PCR was used to clone the cDNA fragment encoding rhSDF-1 alpha P2G. Based on the N-terminal sequence of SEQ ID NOs: 4, 6, 8, and 10, the following specific primers were synthesized:

For SEQ ID NO: 4,
primer 1:
(SEQ ID NO: 23)
acccatgggtcatcatcatcatcatcatgcggcaatgaagggcgtgag
cctgtctta, forward;
and,
primer 2:
(SEQ ID NO: 24)
acaagcttgaattcctactatttgttcagcgc, reverse;
For SEQ ID NO: 6,
primer 3:
(SEQ ID NO: 25)
gaattcatcgaaggtcgtaaaccgg, forward;
and,
primer 4:
(SEQ ID NO: 26)
gcggccgcctactatttgttcagcgctttttc, reverse;
For SEQ ID NO: 8,
primer 5:
(SEQ ID NO: 27)
gagctcgaattcgatgatgatgataaaaaaccggtgagcct,
forward;
primer 6:
(SEQ ID NO: 28)
ctcgaggcggccgcctactatttgttcagcgctttttc, reverse;
For SEQ ID NO: 10,
primer 7:
(SEQ ID NO: 29)
atccatgggctagtaggcaatgaaaccggtgagcctgtctta,
forward;
primer 8:
(SEQ ID NO: 30)
gaattcctactatttgttcagcgc, reverse.

The PCR was performed with each pair of primers using VENT polymerase, and PCR products were size fractionated on a 1.5% agarose gel. The DNA fragments of the correct approximate size were excised from the gel and purified with a Qiaquick Spin Purification Kit (Qiagen, CA). The purified PCR product was ligated with a pPCR-Script Amp SK (+) cloning vectors (Stratagene, CA) in the presence of reaction buffer and ligase at room temperature for about 1 hour. The product was used to transform bacterial DH5a (Invitrogen) competent cells. 2 ml LB was inoculated with a blue colony and grown overnight at 37° C. Cells were harvested by centrifugation (1000 rpm, 5 min, Eppendorf centrifuge, Hamburg) and plasmid DNA was isolated following the manufacturer's protocol (Miniprep Spin Kit, Qiagen, Hilden). Plasmid DNA was eluted into 50 μl water and 5 μl were digested with the corresponding restriction enzymes at the sites used for cloning and ligating. 20 μg of plasmid DNA was sequenced.

### Example 3

Cloning of rhSDF-1alpha P2G cDNA into a Bacterial Expression Vector: rhSDF-1alpha P2G cDNA was prepared by subcloning with NcoI and EcoRI and insertion into a digested pET28a vector as described in Qin et al. (1997), supra, using standard methods. Right clones were verified by restriction enzyme digestion mapping, and the final construct was confirmed by DNA sequencing and alignment analysis. The rhSDF-1alpha P2G cDNA have also been cloned into pTugE07, pGEX4T1, pGEX4T2, pTYB11 and pET31b.

### Example 4

Cloning of rhSDF-1alpha P2G cDNA into a Mammalian Expression Vector: a rhSDF-1alpha P2G gene construct was inserted into pcDNA3.1 (Invitrogen) by ligation. Recombinant plasmids containing the rhSDF-1alpha P2G nucleotides were isolated and confirmed by restriction enzyme digestion and DNA sequencing. Stable cell clones were selected for growth in the presence of G418. Single G418 resistant clones were isolated and shown to contain intact rhSDF-1 alpha P2G cDNA. The clone was used for transient and stable transfection of HEK293 cells by SuperFect (Qiagen) following the vendor's protocol. Clones containing rhSDF-1alpha P2G cDNAs were analyzed for expression using standard immunological techniques, such as Western blotting, immunoprecipitation, and immunofluorescence using antibodies specific to the SDF-1. Isolation of an over-expressing clone with a high copy number of plasmids is accomplished by selecting the clones using increasing doses of the agent.

Cassettes containing rhSDF-1alpha P2G cDNA in the positive orientation, with respect to the promoter, were ligated into appropriate restriction sites 3′ of the promoter and the constructs were confirmed using restriction site mapping and sequencing. These cDNA expression vectors were introduced into fibroblast host cells such as COS-7 (ATCC# CRL1651), CV-1 tat (Sackevitz et al., Science 238: 1575 (1987)), and 293, L (ATCC# CRL6362) by standard methods including, but not limited to, electroporation and chemical procedures (such as cationic liposomes, DEAE dextran, or calcium phosphate). Transfected cells and cell culture supernatants were harvested and analyzed for the expression of rhSDF-1 alpha P2G. The transfection host cells can also include, but are not limited to, CV-1-P (Sackevitz et al., Science 238: 1575 (1987), tk-L (Wigler, et al. Cell 11: 223 (1977)), NS/0, and dHFr-CHO (Kaufman and Sharp, J. Mol. Biol. 159: 601, (1982)).

The vectors used for mammalian transient expression are designed to establish stable cell lines expressing the rhSDF-1alpha P2G. The rhSDF-1alpha P2G cDNA constructs are ligated into vectors containing amplifiable drug-resistance markers for the production of mammalian cell clones that synthesize the highest possible levels of rhSDF-1alpha P2G. Co-transfection of any vector containing rhSDF-1 alpha P2G cDNA can be accomplished using a drug selection plasmid containing, for example, G418, aminoglycoside phosphotransferase; hygromycin, hygromycin-B phosphotransferase; APRT, or xanthine-guanine phosphoribosyl-transferase for the production of stably transfected clones. The clones containing the plasmid are selected using methods well known in the art.

The rhSDF-1alpha P2G can be expressed extracellularly as a secreted protein by ligating rhSDF-1 alpha P2G cDNA constructs to DNA encoding the signal sequence of a secreted protein. The levels of rhSDF-1alpha P2G produced are measured using standard assays.

### Example 5

Cloning of rhSDF-1alpha P2G cDNA into a Baculovirus Expression Vector for Expression in Insect Cells: baculovirus vectors derived from the genome of the AcNPV virus, are designed to provide high level expression of cDNA in the Sf9 line of insect cells (ATCC CRL# 1711). Recombinant baculoviruses expressing the mutant rhSDF-1alpha P2G cDNA are produced by the following standard methods (InVitrogen Maxbac Manual): the rhSDF-1alpha P2G cDNA constructs are ligated into the polyhedrin gene in a variety of baculovirus transfer vectors, including the pAC360 and the BlueBac vector (InVitrogen). Recombinant baculoviruses are generated by homologous recombination following co-transfection of the baculovirus transfer vector and linearized AcNPV genomic DNA (Kitts, P. A., and Nucl. Acid. Res. 18: 5667 (1990)) into Sf9 cells. Recombinant pAC360 viruses are identified by the absence of inclusion bodies in infected cells, and recombinant pBlueBac viruses are identified on the basis of beta-galactosidase expression. Following plaque purification, rhSDF-1alpha P2G expression is measured by standard assays.

The cDNA encoding the entire open reading frame for rhSDF-1alpha P2G is inserted into pBlueBacil. Constructs in the positive orientation are identified by sequence analysis and used to transfect Sf9 cells in the presence of linear AcNPV mild type DNA. Authentic, rhSDF-1alpha P2G is found in the cytoplasm membrane of infected cells. rhSDF-1alpha P2G is extracted from infected cells using methods known in the art (including, for example, hypotonic or detergent lysis).

### Example 6

Cloning of rhSDF-1alpha P2G cDNA into a Yeast Expression Vector: recombinant rhSDF-1alpha P2G is produced in the yeast S. cerevisiae following insertion of the optimal rhSDF-1alpha P2G cDNA cistron into an expression vector designed to direct the intracellular or extracellular expression. In the case of intracellular expression, vectors such as EmBLyex4, or the like, are ligated to the rhSDF-1alpha P2G cistron (see Rinas, U. et al., Biotechnology 8: 543-545 (1990); and Horowitz B. et al., J. Biol. Chem. 265: 4189-4192 (1989). For extracellular expression, the rhSDF-1alpha P2G cistron is ligated into yeast expression vectors which fuse a secretion signal (a yeast or mammalian peptide) to the NH₂ terminus of the rhSDF-1alpha P2G protein (Jacobson, M. A., Gene 85: 511-516 (1989) and Rieft L. and Bellon N. Biochem. 28: 2941-2949 (1989)). These vectors include, but are not limited to, pAVE1.6, which fuses the human serum albumin signal to the expressed cDNA (Steep 0. Biotechnology 8: 42-46 (1990), and the vector pL8PL which fuses the human lysozyme signal to the expressed cDNA (Yamamoto, Y., Biochem. 28: 2728-2732). In addition, rhSDF-1alpha P2G is expressed in yeast as a fusion protein conjugated to ubiquitin using the vector pVEP (see Ecker, D. J., J. Biol. Chem. 264: 7715-7719 (1989), Sabin, E. A., Biotechnology 7: 705-709 (1989), and McDonnell D. P., Mol. Cell. Biol. 9: 5517-5523 (1989). The levels of expressed rhSDF-1alpha P2G are determined using standard assays.

### Example 7

Production of Recombinant rhSDF-1 P2G: rhSDF-1 P2G, both alpha and beta, has been successfully produced in E. coli, purified by affinity chromatography, and released from the affinity tag (in this case a His-tag) by a cleavage reaction using cyanogen bromide. The cloning procedure taught in Example 3 is used.

Expression system: expression of the fusion protein 6H is (SEQ ID NO:34)-rhSDF-1 P2G in inclusion bodies in E. coli is obtained for further isolation on an IMAC column. The rhSDF-1 P2G is released from the fusion protein by chemical cleavage using cyanogen bromide (CNBr). Optimal conditions for expressing the insoluble 6H is (SEQ ID NO:34)-SDF1 fusion protein: induction with 1 mM IPTG in 2YT medium at OD600 1.2-1.4 for 16-18 h (overnight) at 37° C. provides a yield of 300-500 mg of the 6H is (SEQ ID NO:34)-SDF1 fusion protein/L in the E. coli culture.

Lysis/Sulfitolysis/PEI Flocculation: chemical lysis occurs in a 8M urea-based solution (containing sodium tetrathionate and sodium sulfite to cap reactive SH— groups on cysteins) is used to lyse cells by overnight incubation. The overnight incubation is followed by precipitation of bacterial DNA and DNA-related proteins using polyethyleneimine (PEI), and the precipitation is clarified by centrifugation. The supernatant is loaded on a Ni-chelating column for capture.

Fusion protein capture on a Ni-chelating IMAC column: the 6H is (SEQ ID NO:34)-SDF1 fusion protein is captured and purified on a Profinity (Bio-Rad) Ni-chelating column using a 10-400 mM Imidazole gradient over 10 column volumes (CV-20 ml) under denaturing conditions (8M Urea-based buffers). Fractions containing the 6H is (SEQ ID NO:34)-rhSDF-1alpha P2G fusion protein are pooled for loading on a reversed-phase (RP) column.

Purification of 6H is (SEQ ID NO:34)-rhSDF-1alpha P2G on a RP column to remove urea and IMAC buffer salts: the 6H is (SEQ ID NO:34)-rhSDF-1 P2G fusion protein is purified on a RPC15 column (GE) with 0-100% acetonitrile (0.05% TFA) gradient over 10 column volumes (CV-10 ml). Fractions containing the 6H is (SEQ ID NO:34)-rhSDF-1 P2G fusion protein are pooled and lyophilized prior to CNBr cleavage.

CNBr cleavage of 6H is (SEQ ID NO:34)-rhSDF-1alpha P2G fusion protein to release SDF1: the lyophilized 6H is (SEQ ID NO:34)-rhSDF-1 alpha P2G fusion protein is solubilized in 50% formic acid. CNBr is added to a final 1M concentration (115-fold molar excess). The cleavage reaction mixture is incubated in the dark at room temperature for 18-20 h (overnight) under a nitrogen flush. After overnight incubation, most of the formic acid and CNBr is removed by diluting the reaction mixture with a 10-fold volume of water followed by evaporation of the mixture in a rotoevaporator to complete dryness in order to remove traces of CNBr.

Removal of 6H is tag (SEQ ID NO:34) and uncleaved 6H is (SEQ ID NO:34)-rhSDF-1 P2G fusion protein on Ni-chelating column: a dried post-CNBr cleavage pellet is solubilized in a IMAC binding buffer, pH adjusted to 8.0, and then loaded on a Profinity IMAC column. The rhSDF-1 P2G is collected in the flow through (FT) fraction, while 6H is tag (SEQ ID NO:34) and uncleaved 6H is (SEQ ID NO:34)-rhSDF-1 P2G fusion protein binds to the column.

Reduction and refolding of rhSDF-1 P2G: reduction of the rhSDF-1 P2G is accomplished by adding dithiothreitol (DTT) to final 20 mM concentration and incubating the mixture at room temperature for 90 min. Refolding of the reduced rhSDF-1 P2G is accomplished by dialysis in a decreasing urea concentration (4M to 2M to no urea) in 20 mM Tris-HCl buffer pH 8.0. After dialysis, a small amount of precipitate is removed by centrifugation. The supernatant (soluble rhSDF-1 P2G) is tested for protein concentration and submitted for biological activity testing. The supernatant is stored frozen at −20° C.

Characterization of recombinant hSDF-1 P2G: Western blotting detects about ˜0.5 μg using primary monoclonal mouse anti-human SDF1 mAbs (1:5,000) with secondary polyclonal rabbit anti-mouse IgG-AP (1:10,000). The purified, post-CNBr cleavage rhSDF-1 P2G shows a correct 7923.3433 Dalton mass by MALDI-TOF MS, the N-terminal amino acid sequencing correctly shows the sequence Lys-Gly-Val-Ser-Leu (residues 1-5 of SEQ ID NOs:1 and 3).

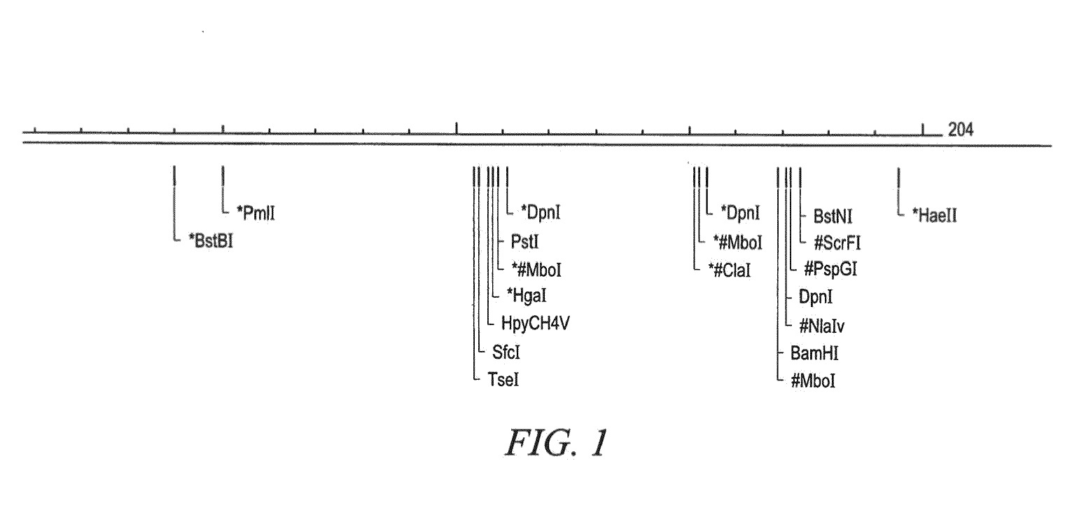

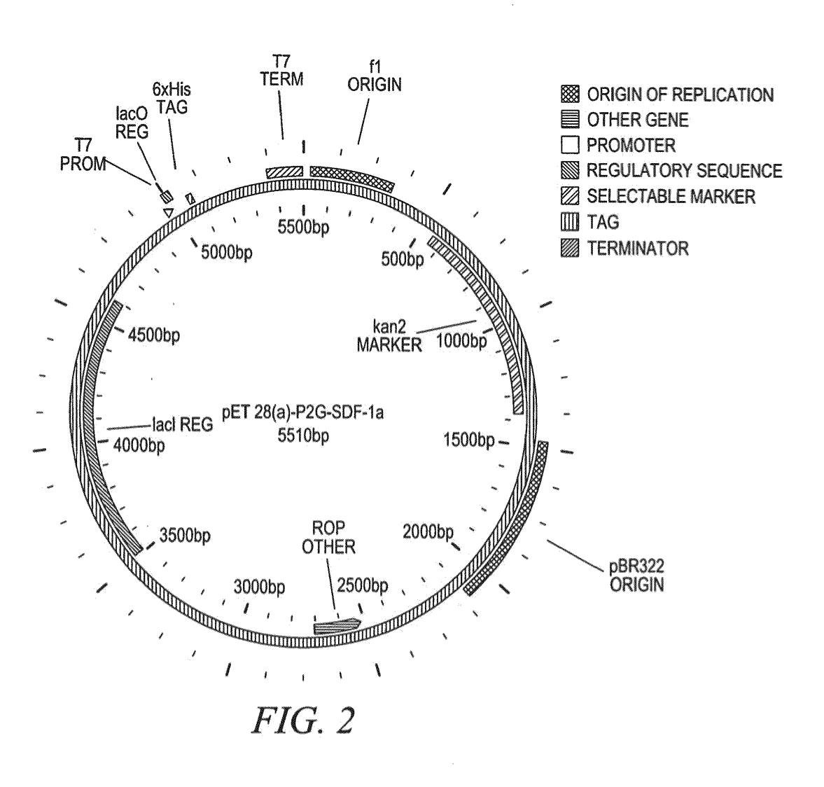

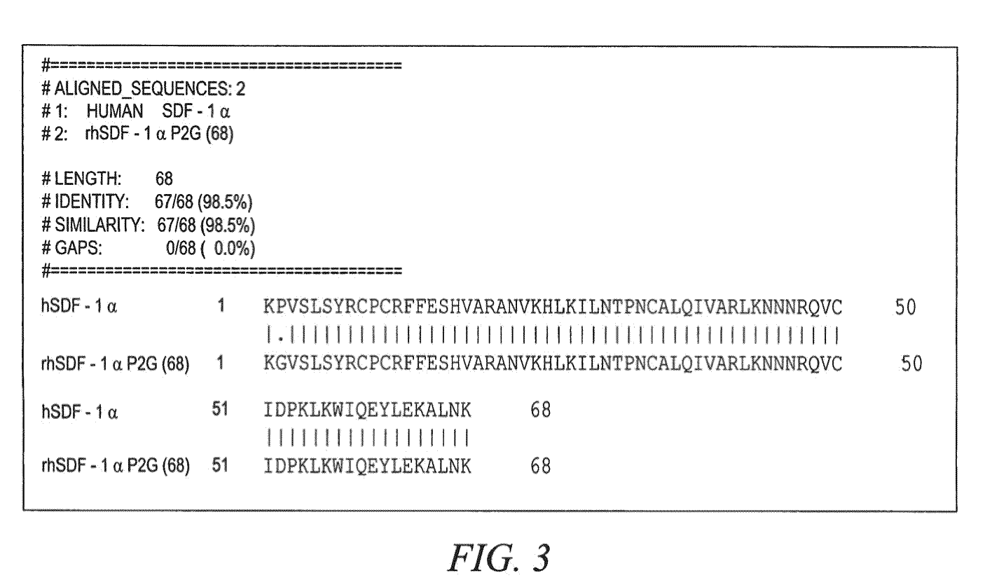

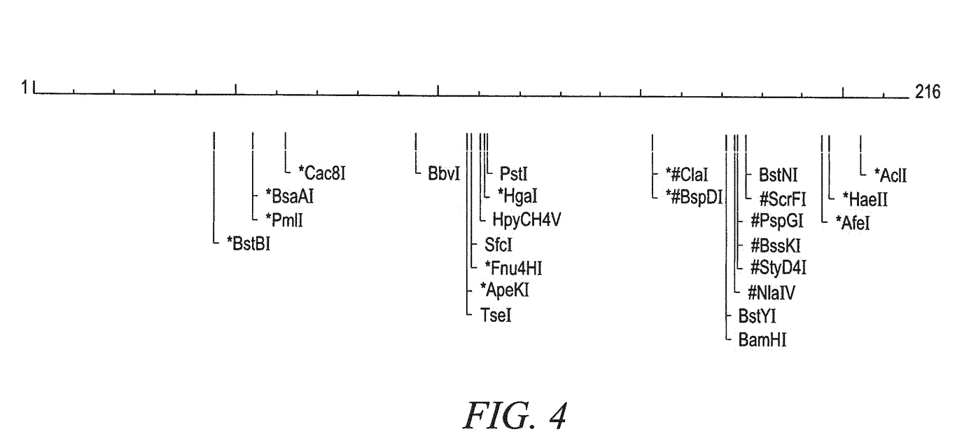

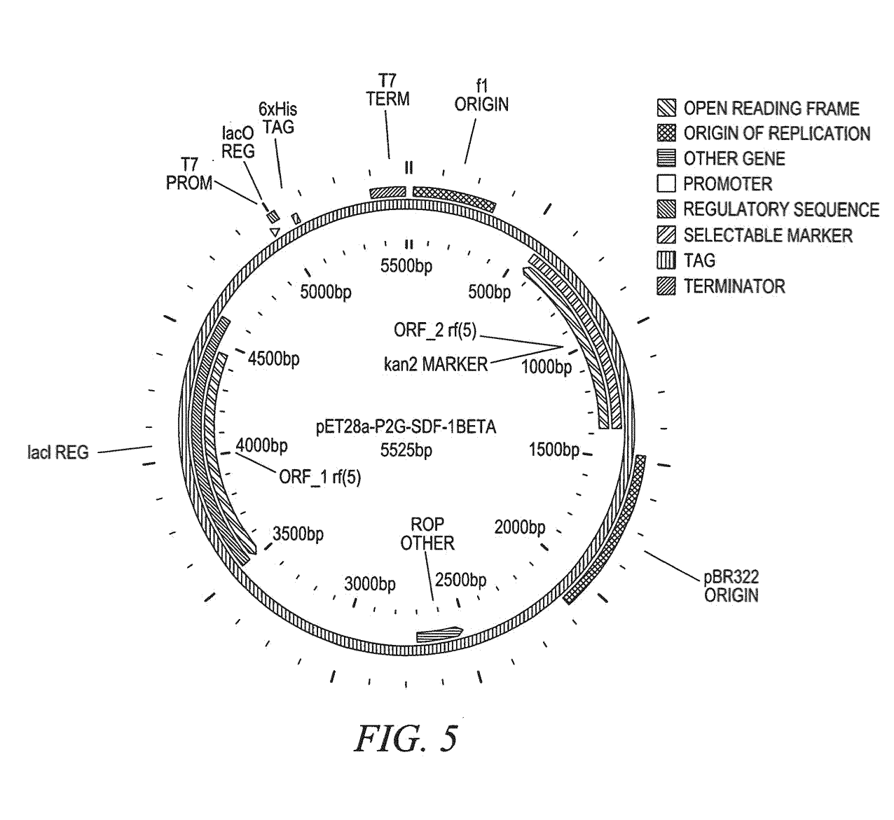

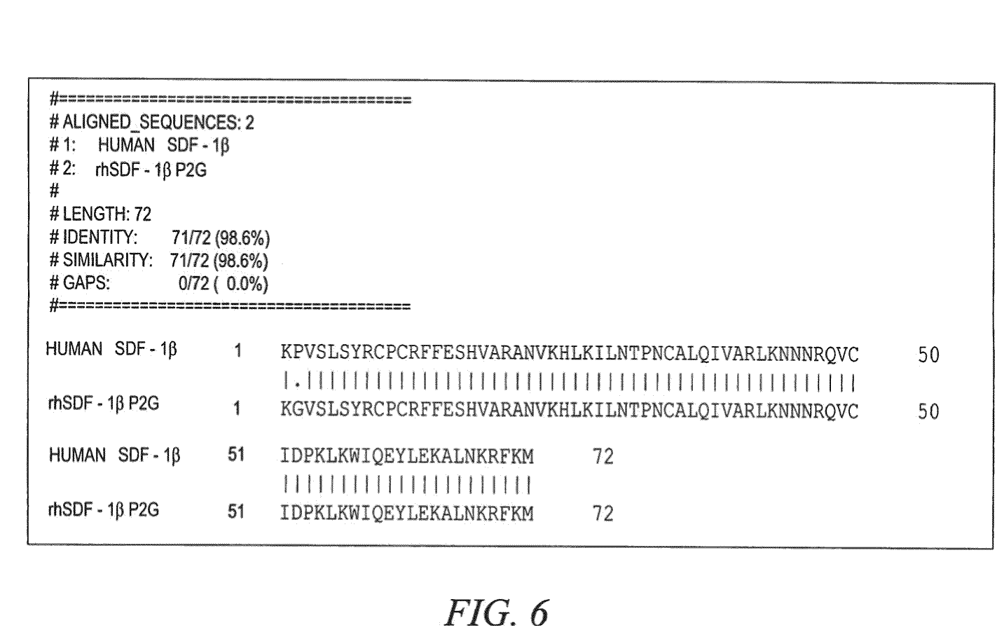

FIG. 1 illustrates the restriction enzyme map of the synthetic DNA for rhSDF-1alpha P2G according to some embodiments of the present invention. FIG. 2 illustrates the plasmid map of the expression vector for rhSDF-1alpha P2G (6×His tag disclosed as SEQ ID NO:34) according to some embodiments of the present invention. FIG. 3 illustrates an amino acid alignment of rhSDF-1alpha P2G (residues 1-68 of SEQ ID NO:3) with human native mature SDF-1alpha (SEQ ID NO:31) according to some embodiments of the present invention. FIG. 4 illustrates the restriction enzyme map of the synthetic DNA for rhSDF-1 beta P2G according to some embodiments of the present invention. FIG. 5 illustrates the plasmid map of the expression vector for rhSDF-1beta P2G (6×His tag disclosed as SEQ ID NO:34) according to some embodiments of the present invention. FIG. 6 illustrates an amino acid alignment of rhSDF-1beta P2G (SEQ ID NO:36) with human native mature SDF-1 beta (SEQ ID NO:35) according to some embodiments of the present invention.

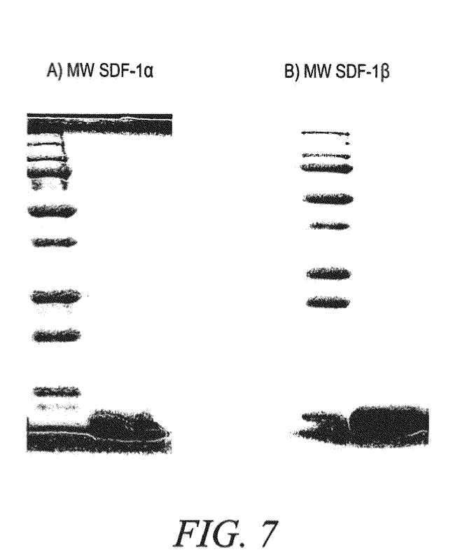

FIGS. 7A and 7B show purification of the rhSDF-1alpha P2G and rhSDF-1beta P2G, respectively, using Coomassie Blue staining and 15% SDS-PAGE analyses according to some embodiments of the present invention. FIG. 7A shows purified rhSDF-1 alpha P2G in an amount of about 1 μg, and FIG. 7B shows purified rhSDF-1 beta P2G in an amount of about 3 μg. The molecular weight scale in the gel sections provided on the left of each figure are, from top to bottom, 250, 150, 100, 75, 50, 37, 25, 20, 15, and 10 kDaltons.

### Example 8

The binding affinity of rhSDF-1alpha P2G was determined by a radioactive ligand binding assay. The efficacy of the rhSDF-1 alpha P2G at binding to two different types of cells having either CXCR4 receptors or CXCR7 receptors was measured.

Binding Assay: the following procedures are carried out at 4° C. Cells with rhSDF-1alpha P2G are washed with PBS and suspended in lysis buffer (10 mM Tris-HCl pH7.5, 2 mM EDTA and proteinase inhibitor cocktail). The cells are incubated on ice for 40 minutes followed by a brief sonication. The cell debris is removed by centrifugation at 1000 rpm for 10 minutes, and the supernatant is centrifuged for 1 hour at 50,000 rpm. The pellet is resuspended in the lysis buffer and kept at 80° C.

The experiments include contacting a cell with rhSDF-1alpha P2G. The experiments were performed using two different cell types to show CXCR4 and CXCR7 binding affinity: a human acute lymphoblastic T-cell leukemia (CEM) cell line to measure CXCR4 binding affinity, a, and an RDC-1 cell line (COS-1) to measure CXCR7 binding affinity.

The cell lines were used at a concentration of about 5×10⁶ cells/ml, an amount adjusted according to procedures known to one of skill. A DURAPORE membrane and a Millipore MultiScreen 96-well plate were used in the binding assay, and the membrane was blocked with a PVP/Tween-based blocking buffer before use. An RPMI-based binding buffer, 0-400 nM of SDF-1 or 0-400 μM of an SDF-1 mimetic, a competitive dose of 0.02 nM ¹²⁵I-SDF-1 (Amersham), and the desired cell line was added to the wells for each experiment. The cells were incubated at 4° C. with shaking for 2 h, followed by triplicate washes with PBS. Bound ¹²⁵I-SDF-1 was counted using a CliniGamma gamma counter (LKB Wallac). Experiments were performed in triplicate, and competition curves were fitted after subtracting non-specific binding to both filters and cells.

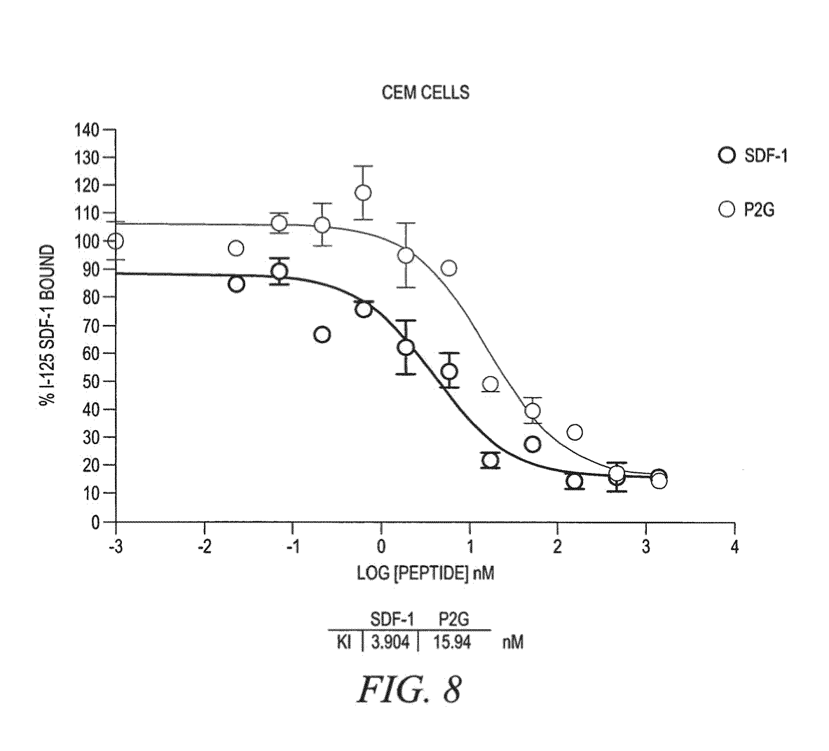

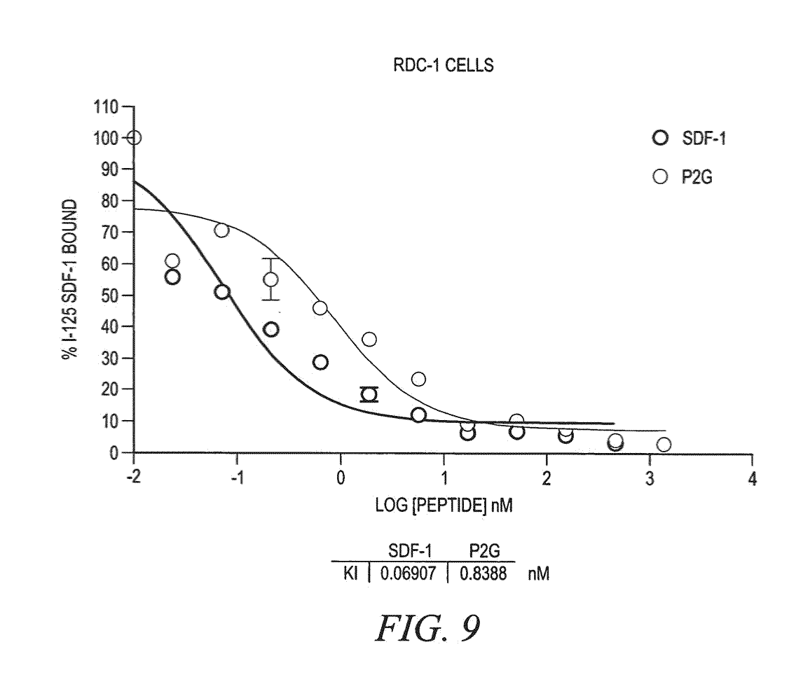

The results are expressed as Ki values in FIGS. 8 and 9. FIG. 8 shows competitive CXCR4 receptor binding between SDF-1 and rhSDF-1alpha P2G on a CEM cell line according to some embodiments of the present invention. FIG. 9 shows competitive CXCR4 receptor binding between SDF-1 and rhSDF-1alpha P2G on an RDF-1 cell line according to some embodiments of the present invention.

### Example 9

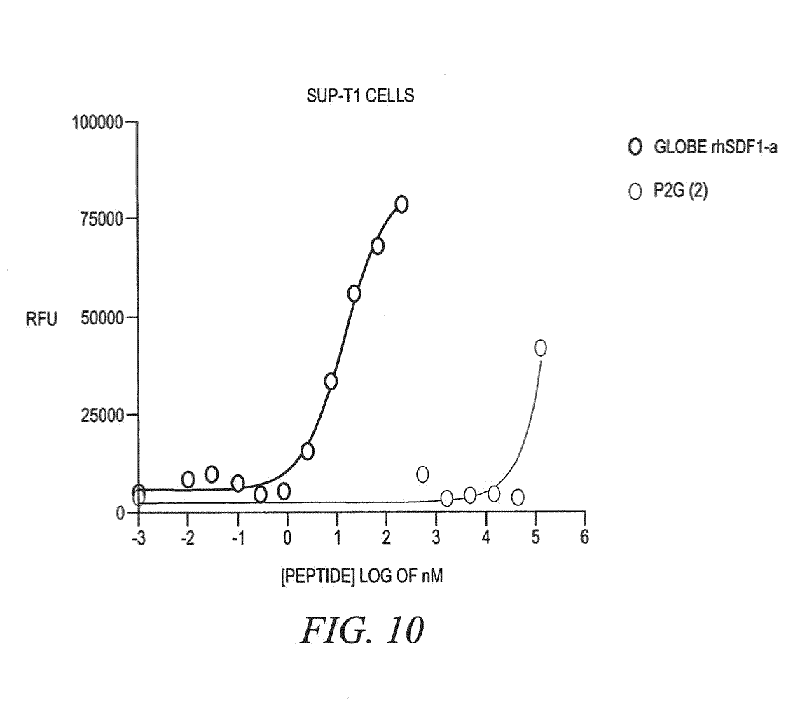

FIG. 10 shows the effect of induction of calcium mobilization by rhSDF-1alpha P2G analogs at a concentration of 1 μm according to some embodiments of the present invention. The results show that the rhSDF-1 bind to both CXCR4 and CXCR7 receptors and compete effectively with SDF-1.

A human T-cell lymphoma (SUP-T1) cell line was used to measure the activation of a CXCR4 receptor by both an rhSDF-1alpha and an rhSDF-1alpha P2G analog. The results in FIG. 9 shows the calcium mobilization achieved by increasing amounts of rhSDF-1alpha and, comparatively, that a concentration of 1 μM of the rhSDF-1alpha P2G is a strong inhibitor of the calcium mobilization created by the rhSDF-1alpha at the same increasing concentrations.

### Example 10

This example illustrates the selectivity of the rhSDF-1alpha-P2G analogs at receptor binding and mediating intracellular calcium mobilization.

A suspension of CXCR-3/300-19 cells, which are mouse pre-B lymphocytes transfected with the CXCR3 receptor, (Moser, et al), were washed in RPMI media, resuspended in RPMI media supplemented with 10% FCS, and then plated at 1.2×10⁵ cells/well in a 96-well black wall/clear bottom plates coated with poly-D-lysine (Becton Dickinson). The plates were loaded with 100 μL fluorescent calcium indicator FLIPR Calcium 3 assay kit component A (Molecular Probes) for 1 hr at 37° C. The cells were then spun on the plates at 1000 rpm for 15 minutes at room temperature. The intracellular calcium mobilization in response to 25 μL (0-100000 nM final concentration) of the analogue at various concentrations was measured at 37° C. by monitoring the fluorescence as a function of time in all of the wells using a Flexstation Fluorometric Imaging Plate Reader (Molecular Devices). All analogues were run simultaneously with rhIP-10 (R&D Systems) as the standard to show specificity of the rhSDF-1 alpha P2G analog. The results showed a complete lack of effect of the rhSDF-1 alpha P2G analog on reducing the induction of binding and calcium mobilization by the rhIP-10 on a CXCR3 receptor, suggesting a high selectivity of the rhSDF-1alpha P2G analog for the CXCR4 and CXCR7 receptors.

Those skilled in the art will recognize, or be able to ascertain using no more than routine experimentation, that there are many equivalents to the specific embodiments described herein that have been described and enabled to the extent that one of skill in the art can practice the invention well-beyond the scope of the specific embodiments taught herein. Such equivalents are intended to be encompassed by the following claims. In addition, there are numerous lists and Markush groups taught and claimed herein. One of skill will appreciate that each such list and group contains various species and can be modified by the removal, or addition, of one or more of species, since every list and group taught and claimed herein may not be applicable to every embodiment feasible in the practice of the invention. As such, components in such lists can be removed and are expected to be removed to reflect some embodiments taught herein. All publications, patents, and patent applications mentioned in this application are herein incorporated by reference into the specification to the same extent as if each was specifically indicated to be herein incorporated by reference in its entirety.

We claim:

1. A method of treating a solid tumor in a mammal comprising administering an effective amount of an isolated recombinant polypeptide to the mammal, the peptide comprising:
SEQ ID NO:1 produced by capturing and purifying a fusion polypeptide having an affinity tag, and removing the affinity tag for a high yield production of the SEQ ID NO:1; or
an isolated recombinant polypeptide comprising a peptide sequence, wherein said peptide sequence is at least 95% homologous to SEQ ID NO:1, and conserves the Gly at residue position number 2 of SEQ ID NO:1;
wherein the isolated recombinant polypeptide binds to a CXCR7 receptor.

2. The method of claim 1, wherein the fusion peptide comprises:
the sequence Lys-Arg-Phe-Lys (SEQ ID NO: 33) at the C-terminus of SEQ ID NO:1 to provide a polypeptide comprising SEQ ID NO:3; or
a peptide sequence that is at least 95% homologous to SEQ ID NO:3, conserves the Gly at residue position number 2 of SEQ ID NO:3, and binds to a CXCR7 receptor.

3. The method of claim 1, wherein the fusion polypeptide comprises an amino acid sequence selected from the group consisting of SEQ ID NOs:5, 7, 9, and 11.

4. The method of claim 2, wherein the fusion polypeptide comprises comprises an amino acid sequence selected from the group consisting of SEQ ID NOs:14, 16, 18, and 20.

5. The method of claim 1, wherein the solid tumor is lung carcinoma.

6. The method of claim 1, wherein the treating includes inhibiting angiogenesis.

7. The method of claim 1, further comprising administering an effective amount of interferon beta.

8. A method of inhibiting interferon gamma production by an activated T-cell comprising contacting the T-cell with an effective amount of an isolated recombinant polypeptide, the peptide comprising:
SEQ ID NO:1 produced by capturing and purifying a fusion polypeptide having an affinity tag, and removing the affinity tag for a high yield production of the SEQ ID NO:1; or
an isolated recombinant polypeptide comprising a peptide sequence, wherein said peptide sequence is at least 95% homologous to SEQ ID NO:1, and conserves the Gly at residue position number 2 of SEQ ID NO:1; wherein the isolated recombinant polypeptide binds to a CXCR7 receptor.

9. The method of claim 8, wherein the fusion peptide comprises:
the sequence Lys-Arg-Phe-Lys (SEQ ID NO: 33) at the C-terminus of SEQ ID NO:1 to provide a polypeptide comprising SEQ ID NO:3; or
a peptide sequence that is at least 95% homologous to SEQ ID NO:3, conserves the Gly at residue position number 2 of SEQ ID NO:3, and binds to a CXCR7 receptor.

10. The method of claim 8, wherein the fusion polypeptide comprises an amino acid sequence selected from the group consisting of SEQ ID NOs:5, 7, 9, and 11.

11. The method of claim 9, wherein the fusion polypeptide comprises comprises an amino acid sequence selected from the group consisting of SEQ ID NOs:14, 16, 18, and 20.

12. The method of claim 8, wherein the activated T-cell is a human T-lymphoma cell.

13. The method of claim 8 further comprising contacting the activated T-cell with interferon beta to provide a synergistic down-regulation of interferon gamma production.

14. The method of claim 8, wherein the contacting comprises administering to a subject a therapeutically effective amount of the polypeptide of claim 1 in a pharmaceutically acceptable carrier.

15. The method of claim 14, wherein the administering is in an amount sufficient to (i) reduce the onset of, (ii) reduce the progress of, and/or (iii) reverse symptoms of multiple sclerosis.

16. A method of increasing hematopoietic cell proliferation in a subject by administering to the subject a therapeutically effective amount of the polypeptide of claim 1 in a pharmaceutically acceptable carrier.

17. The method of claim 16, wherein the fusion peptide comprises:
the sequence Lys-Arg-Phe-Lys (SEQ ID NO: 33) at the C-terminus of SEQ ID NO:1 to provide a polypeptide comprising SEQ ID NO:3; or
a peptide sequence that is at least 95% homologous to SEQ ID NO:3, conserves the Gly at residue position number 2 of SEQ ID NO:3, and binds to a CXCR7 receptor.

18. The method of claim 16, wherein the fusion polypeptide comprises an amino acid sequence selected from the group consisting of SEQ ID NOs:5, 7, 9, and 11.

19. The method of claim 18, wherein the fusion polypeptide comprises comprises an amino acid sequence selected from the group consisting of SEQ ID NOs:14, 16, 18, and 20.

20. The method of claim 16, wherein the hematopoietic cell is a bone marrow progenitor cell.


SEQUENCE LISTING (36 sequences)

SEQ ID NO: 1 - protein, 67 aa, Artificial Sequence
  Description of Artificial Sequence Synthetic polypeptide
KGVSLSYRCPCRFFESHVARANVKHLKILNTPNCALQIVARLKNNNRQVCIDPKLKWIQEYLEKALN

SEQ ID NO: 2 - DNA, 204 bp, Artificial Sequence
  Description of Artificial Sequence Synthetic polynucleotide
aagggcgtgagcctgtcttatcgttgtccgtgtcgttttttcgaaagccacgtggcgcgtgcgaacgtgaaacacctgaaaatcctgaacaccccgaactgtgcgctgcagatcgtggcgcgtctgaaaaacaacaaccgtcaggtgtgtatcgatccgaaactgaaatggatccaggaatatctggaaaaagcgctgaactag

SEQ ID NO: 3 - protein, 71 aa, Artificial Sequence
  Description of Artificial Sequence Synthetic polypeptide
KGVSLSYRCPCRFFESHVARANVKHLKILNTPNCALQIVARLKNNNRQVCIDPKLKWIQEYLEKALNKRFK

SEQ ID NO: 4 - DNA, 216 bp, Artificial Sequence
  Description of Artificial Sequence Synthetic polynucleotide
aagggcgtgagcctgtcttatcgttgtccgtgtcgttttttcgaaagccacgtggcgcgtgcgaacgtgaaacacctgaaaatcctgaacaccccgaactgtgcgctgcagatcgtggcgcgtctgaaaaacaacaaccgtcaggtgtgtatcgatccgaaactgaaatggatccaggaatatctggaaaaagcgctgaacaaacgtttcaagtag

SEQ ID NO: 5 - protein, 78 aa, Artificial Sequence
  Description of Artificial Sequence Synthetic polypeptide
MGHHHHHHAAMKGVSLSYRCPCRFFESHVARANVKHLKILNTPNCALQIVARLKNNNRQVCIDPKLKWIQEYLEKALN

SEQ ID NO: 6 - DNA, 237 bp, Artificial Sequence
  Description of Artificial Sequence Synthetic polynucleotide
atgggtcatcatcatcatcatcatgcggcaatgaagggcgtgagcctgtcttatcgttgtccgtgtcgttttttcgaaagccacgtggcgcgtgcgaacgtgaaacacctgaaaatcctgaacaccccgaactgtgcgctgcagatcgtggcgcgtctgaaaaacaacaaccgtcaggtgtgtatcgatccgaaactgaaatggatccaggaatatctggaaaaagcgctgaactag

SEQ ID NO: 7 - protein, 107 aa, Artificial Sequence
  Description of Artificial Sequence Synthetic polypeptide
MGSSHHHHHHSSGLVPRGSHMASMTGGQQMGRGSEFIEGRKGVSLSYRCPCRFFESHVARANVKHLKILNTPNCALQIVARLKNNNRQVCIDPKLKWIQEYLEKALN

SEQ ID NO: 8 - DNA, 324 bp, Artificial Sequence
  Description of Artificial Sequence Synthetic polynucleotide
atgggcagcagccatcatcatcatcatcacagcagcggcctggtgccgcgcggcagccatatggctagcatgactggtggacagcaaatgggtcgcggatccgaattcatcgaaggtcgtaagggcgtgagcctgtcttatcgttgtccgtgtcgttttttcgaaagccacgtggcgcgtgcgaacgtgaaacacctgaaaatcctgaacaccccgaactgtgcgctgcagatcgtggcgcgtctgaaaaacaacaaccgtcaggtgtgtatcgatccgaaactgaaatggatccaggaatatctggaaaaagcgctgaactag

SEQ ID NO: 9 - protein, 108 aa, Artificial Sequence
  Description of Artificial Sequence Synthetic polypeptide
MGSSHHHHHHSSGLVPRGSHMASMTGGQQMGRGSEFDDDDKKGVSLSYRCPCRFFESHVARANVKHLKILNTPNCALQIVARLKNNNRQVCIDPKLKWIQEYLEKALN

SEQ ID NO: 10 - DNA, 327 bp, Artificial Sequence
  Description of Artificial Sequence Synthetic polynucleotide
atgggcagcagccatcatcatcatcatcacagcagcggcctggtgccgcgcggcagccatatggctagcatgactggtggacagcaaatgggtcgcggatccgaattcgatgatgatgataaaaagggcgtgagcctgtcttatcgttgtccgtgtcgttttttcgaaagccacgtggcgcgtgcgaacgtgaaacacctgaaaatcctgaacaccccgaactgtgcgctgcagatcgtggcgcgtctgaaaaacaacaaccgtcaggtgtgtatcgatccgaaactgaaatggatccaggaatatctggaaaaagcgctgaactag

SEQ ID NO: 11 - protein, 68 aa, Artificial Sequence
  Description of Artificial Sequence Synthetic polypeptide
MKGVSLSYRCPCRFFESHVARANVKHLKILNTPNCALQIVARLKNNNRQVCIDPKLKWIQEYLEKALN

SEQ ID NO: 12 - DNA, 207 bp, Artificial Sequence
  Description of Artificial Sequence Synthetic polynucleotide
atgaagggcgtgagcctgtcttatcgttgtccgtgtcgttttttcgaaagccacgtggcgcgtgcgaacgtgaaacacctgaaaatcctgaacaccccgaactgtgcgctgcagatcgtggcgcgtctgaaaaacaacaaccgtcaggtgtgtatcgatccgaaactgaaatggatccaggaatatctggaaaaagcgctgaactag

SEQ ID NO: 13 - DNA, 5510 bp, Artificial Sequence
  Description of Artificial Sequence Synthetic polynucleotide
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggtcatcatcatcatcatcatgcggcaatgaagggcgtgagcctgtcttatcgttgtccgtgtcgttttttcgaaagccacgtggcgcgtgcgaacgtgaaacacctgaaaatcctgaacaccccgaactgtgcgctgcagatcgtggcgcgtctgaaaaacaacaaccgtcaggtgtgtatcgatccgaaactgaaatggatccaggaatatctggaaaaagcgctgaacaaatagtaggaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 14 - protein, 82 aa, Artificial Sequence
  Description of Artificial Sequence Synthetic polypeptide
MGHHHHHHAAMKGVSLSYRCPCRFFESHVARANVKHLKILNTPNCALQIVARLKNNNRQVCIDPKLKWIQEYLEKALNKRFK

SEQ ID NO: 15 - DNA, 249 bp, Artificial Sequence
  Description of Artificial Sequence Synthetic polynucleotide
atgggtcatcatcatcatcatcatgcggcaatgaagggcgtgagcctgtcttatcgttgtccgtgtcgttttttcgaaagccacgtggcgcgtgcgaacgtgaaacacctgaaaatcctgaacaccccgaactgtgcgctgcagatcgtggcgcgtctgaaaaacaacaaccgtcaggtgtgtatcgatccgaaactgaaatggatccaggaatatctggaaaaagcgctgaacaaacgtttcaagtag

SEQ ID NO: 16 - protein, 111 aa, Artificial Sequence
  Description of Artificial Sequence Synthetic polypeptide
MGSSHHHHHHSSGLVPRGSHMASMTGGQQMGRGSEFIEGRKGVSLSYRCPCRFFESHVARANVKHLKILNTPNCALQIVARLKNNNRQVCIDPKLKWIQEYLEKALNKRFK

SEQ ID NO: 17 - DNA, 336 bp, Artificial Sequence
  Description of Artificial Sequence Synthetic polynucleotide
atgggcagcagccatcatcatcatcatcacagcagcggcctggtgccgcgcggcagccatatggctagcatgactggtggacagcaaatgggtcgcggatccgaattcatcgaaggtcgtaagggcgtgagcctgtcttatcgttgtccgtgtcgttttttcgaaagccacgtggcgcgtgcgaacgtgaaacacctgaaaatcctgaacaccccgaactgtgcgctgcagatcgtggcgcgtctgaaaaacaacaaccgtcaggtgtgtatcgatccgaaactgaaatggatccaggaatatctggaaaaagcgctgaacaaacgtttcaagtag

SEQ ID NO: 18 - protein, 112 aa, Artificial Sequence
  Description of Artificial Sequence Synthetic polypeptide
MGSSHHHHHHSSGLVPRGSHMASMTGGQQMGRGSEFDDDDKKGVSLSYRCPCRFFESHVARANVKHLKILNTPNCALQIVARLKNNNRQVCIDPKLKWIQEYLEKALNKRFK

SEQ ID NO: 19 - DNA, 339 bp, Artificial Sequence
  Description of Artificial Sequence Synthetic polynucleotide
atgggcagcagccatcatcatcatcatcacagcagcggcctggtgccgcgcggcagccatatggctagcatgactggtggacagcaaatgggtcgcggatccgaattcgatgatgatgataaaaagggcgtgagcctgtcttatcgttgtccgtgtcgttttttcgaaagccacgtggcgcgtgcgaacgtgaaacacctgaaaatcctgaacaccccgaactgtgcgctgcagatcgtggcgcgtctgaaaaacaacaaccgtcaggtgtgtatcgatccgaaactgaaatggatccaggaatatctggaaaaagcgctgaacaaacgtttcaagtag

SEQ ID NO: 20 - protein, 72 aa, Artificial Sequence
  Description of Artificial Sequence Synthetic polypeptide
MKGVSLSYRCPCRFFESHVARANVKHLKILNTPNCALQIVARLKNNNRQVCIDPKLKWIQEYLEKALNKRFK

SEQ ID NO: 21 - DNA, 219 bp, Artificial Sequence
  Description of Artificial Sequence Synthetic polynucleotide
atgaagggcgtgagcctgtcttatcgttgtccgtgtcgttttttcgaaagccacgtggcgcgtgcgaacgtgaaacacctgaaaatcctgaacaccccgaactgtgcgctgcagatcgtggcgcgtctgaaaaacaacaaccgtcaggtgtgtatcgatccgaaactgaaatggatccaggaatatctggaaaaagcgctgaacaaacgtttcaagtag

SEQ ID NO: 22 - DNA, 5525 bp, Artificial Sequence
  Description of Artificial Sequence Synthetic polynucleotide
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggtcatcatcatcatcatcatgcggcaatgaagggcgtgagcctgtcttatcgttgtccgtgtcgttttttcgaaagccacgtggcgcgtgcgaacgtgaaacacctgaaaatcctgaacaccccgaactgtgcgctgcagatcgtggcgcgtctgaaaaacaacaaccgtcaggtgtgtatcgatccgaaactgaaatggatccaggaatatctggaaaaagcgctgaacaaacgtttcaagtagtagggatccgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 23 - DNA, 57 bp, Artificial Sequence
  Description of Artificial Sequence Synthetic primer
acccatgggtcatcatcatcatcatcatgcggcaatgaagggcgtgagcctgtctta

SEQ ID NO: 24 - DNA, 32 bp, Artificial Sequence
  Description of Artificial Sequence Synthetic primer
acaagcttgaattcctactatttgttcagcgc

SEQ ID NO: 25 - DNA, 25 bp, Artificial Sequence
  Description of Artificial Sequence Synthetic primer
gaattcatcgaaggtcgtaaaccgg

SEQ ID NO: 26 - DNA, 32 bp, Artificial Sequence
  Description of Artificial Sequence Synthetic primer
gcggccgcctactatttgttcagcgctttttc

SEQ ID NO: 27 - DNA, 41 bp, Artificial Sequence
  Description of Artificial Sequence Synthetic primer
gagctcgaattcgatgatgatgataaaaaaccggtgagcct

SEQ ID NO: 28 - DNA, 38 bp, Artificial Sequence
  Description of Artificial Sequence Synthetic primer
ctcgaggcggccgcctactatttgttcagcgctttttc

SEQ ID NO: 29 - DNA, 42 bp, Artificial Sequence
  Description of Artificial Sequence Synthetic primer
atccatgggctagtaggcaatgaaaccggtgagcctgtctta

SEQ ID NO: 30 - DNA, 24 bp, Artificial Sequence
  Description of Artificial Sequence Synthetic primer
gaattcctactatttgttcagcgc

SEQ ID NO: 31 - protein, 68 aa, Homo sapiens
KPVSLSYRCPCRFFESHVARANVKHLKILNTPNCALQIVARLKNNNRQVCIDPKLKWIQEYLEKALNK

SEQ ID NO: 32 - protein, 72 aa, Homo sapiens
KPVSLSYRCPCRFFESHVARANVKHLKILNTPNCALQIVARLKNNNRQVCIDPKLKWIQEYLEKALNKRFKM

SEQ ID NO: 33 - protein, 4 aa, Artificial Sequence
  Description of Artificial Sequence Synthetic peptide
KRFK

SEQ ID NO: 34 - protein, 6 aa, Artificial Sequence
  Description of Artificial Sequence Synthetic 6xHis tag
HHHHHH

SEQ ID NO: 35 - protein, 72 aa, Homo sapiens; identical to SEQ ID NO: 32

SEQ ID NO: 36 - protein, 72 aa, Artificial Sequence
  Description of Artificial Sequence Synthetic polypeptide
KGVSLSYRCPCRFFESHVARANVKHLKILNTPNCALQIVARLKNNNRQVCIDPKLKWIQEYLEKALNKRFKM